(19) United States
(12) Patent Application Publication
(10) Pub. No.: US 2016/0060703 A1
(43) Pub. Date: Mar. 3, 2016

(54) HAPLOTYPE DETECTION

(71) Applicant: UNIVERSITY OF SOUTHAMPTON, Southampton Hampshire (GB)

(72) Inventors: Timothy John ELLIOTT, Southampton, Hampshire (GB); Edward Nicholas JAMES, Southampton, Hampshire (GB); Christopher John EDWARDS, Southampton, Hampshire (GB)

(73) Assignee: UNIVERSITY OF SOUTHAMPTON, Southampton Hampshire (GB)

(21) Appl. No.: 14/888,402

(22) PCT Filed: Apr. 30, 2014
(86) PCT No.: PCT/GB2014/051339

§ 371 Date: Oct. 30, 2015

(30) Foreign Application Priority Data
May 1, 2013 (GB) 1307843.1

(51) Int. Cl. C12Q 1/68; G01N 33/573

(52) U.S. Cl. CPC C12Q 1/6883; G01N 33/573; C12Q 2600/172; C12Q 2600/156; C12Q 2600/112; C12Q 2600/106; G01N 2333/948

(57) ABSTRACT

A method of diagnosing Ankylosing Spondylitis (AS), a spondyloarthropathy, arthritis, psoriasis, type-1 diabetes or a carcinoma comprising typing the ERAP1 haplotype of an individual.

### FIELD OF THE INVENTION

The invention relates to determining the presence or absence of a haplotype in the genome of an individual.

### BACKGROUND OF THE INVENTION

Diagnosis of conditions based on determination of genetic susceptibility is increasingly common. Conditions such as spondylarthropathies, psoriasis and cancers have genetic components, and typing of these genetic components can be used in diagnosis.

Ankylosing spondylitis (AS) is a severe inflammatory disease that damages joints with a predilection for the spine. The disease can cause severe pain and disability and is present in around 200,000 people in the United Kingdom. Currently, the diagnosis, prediction of prognosis and decisions on the most appropriate treatment are based on clinical features and composite scores of history, clinical examination, and a number of non-specific blood tests and radiological investigations (X-ray, MRI). There is no specific diagnostic test currently available. This is particularly important when trying to identify patients with AS amongst the large number of individuals presenting to medical attention every year with back pain. This difficulty is highlighted by the experience of many patients with AS who may spend several years with symptoms before a diagnosis is made.

A strong association between the HLA-B27 MHC class I allele and AS has been known for 40 years. Recent studies have revealed numerous single nucleotide polymorphisms (SNP's) within the endoplasmic reticulum (ER) resident aminopeptidase ERAP1.

### SUMMARY OF THE INVENTION

The inventors have identified new ERAP1 haplotypes and investigated how these haplotypes affect disease susceptibility as well as ERAP1 function. Accordingly, the present invention provides a method of diagnosing Ankylosing spondylitis (AS), a spondyloarthropathy, arthritis, psoriasis, type-1 diabetes or a carcinoma comprising typing the ERAP1 haplotype of an individual to determine whether the individual has a hyper or hypo haplotype, wherein said haplotype comprises at least 2 SNP's.

### DESCRIPTION OF THE DRAWINGS

FIG. 1. The trimming activity of identified ERAP1 haplotypes. (A) Erap1-deficient fibroblasts were transfected with X5-SHL8 and WT ERAP1 (▴) or functionally inactive E320A ERAP1 (∘) and assayed for trimming by stimulation of the LacZ-inducible, SHL8/K^{b}-specific B3Z T cell hybridoma. As a control for ERAP1 trimming B6 fibroblasts were transfected with X5-SHL8 only (□) and assayed for trimming as above. (B) Erap1-deficient fibroblasts transfected with X5-SHL8 and WT (□), E320A (∘), or identified ERAP1 haplotypes M349V, K528R, M349V/K528R, M349V/D575N/R725Q, K528R/R725Q, K528R/Q730E, R725Q/Q730E or 5SNP (▴) and were titrated and assayed for trimming as above. Data are representative of six experiments. (C) Erap1-deficient fibroblasts were transfected with ERAP1 haplotypes and X5-SHL8, as above, and the relative maximum B3Z response compared to WT calculated. Bars show results pooled from at least six experiments±SEM (***; P<0.001, ns; not significant). From left to right bars show results for WT, M349V, K528R, M349V/K528R, M349V/D575N/R725Q, K528R/R725Q, K528R/Q730E, R725Q/Q730E and 5SNP

FIG. 2. RP-HPLC analysis of ERAP1 haplotypes reveals hypo- and hyper-active trimming phenotypes. (A-J) Erap1-deficient fibroblasts were transfected with X5-SHL8 and ERAP1 haplotypes () identified from individuals. Peptide extracts from transfected fibroblasts were fractionated by RP-HPLC, pretreated with trypsin to allow detection of N-terminally extended SHL8 analogs and assayed with B3Z T cell hybridoma and H-2K^{b}-L cells as APCs. Retention times of synthetic SHL8, K-SHL8 and X5-SHL8 peptides are marked with arrows. Fractions from runs of buffer alone were assayed in parallel to exclude the possibility of sample carry over between runs (∘). HPLC elution profiles are representative of individual ‘runs’ from four experiments.

FIG. 3. Hyper-active ERAP1 haplotypes destroy peptides by over-trimming. Erap1-deficient fibroblasts were transfected with dt-SHL8 (A) or dt-KSHL8 (B) and empty vector (∘), WT (), 5SNP (▾) or R725Q/Q730E (▴) ERAP1, titrated and assayed for stimulation of B3Z T cell hybridoma as in FIG. 1. (C) Peptides eluted from dt-KSHL8K expressed by Erap1-deficient fibroblasts by trypsin following immunopurification by anti-H-2K^{b} antibody Y3 were fractionated by RP-HPLC. Fractions were untreated (▴) or pretreated with carboxypeptidase B () to allow detection of SHL8 and assayed with B3Z and H-2K^{b}-L cells as APCs. Downward arrow indicates the peak elution of SHL8K. Fractions from mock injection (∘) were performed as in FIG. 2. (D) Peptides eluted from Erap1-deficient fibroblasts transfected with dt-KSHL8K and WT (), vector (▾), 5SNP (▴) or R725Q/Q730E (▪) were fractionated by RP-HPLC as in C. Data are representative of four experiments. (E) Chromatogram of RP-HPLC fractionated peptides eluted from Erap1-deficient fibroblasts transfected as in (D) at mass fragments m/z=1100; SHL8K (left panel) and m/z=1013; IHL7K (right panel). Downward arrows indicate the retention time of SHL8K and IHL7K peptides. Data are representative of three experiments.

FIG. 4. Fine specificity of N-terminal amino acid trimming by ERAP1 haplotypes. (A) Erap1-deficient fibroblasts transfected with X6-SHL8 and WT, E320A, or ERAP1 haplotypes M349V, K528R, M349V/K528R, M349V/D575N/R725Q, K528R/R725Q, K528R/Q730E, R725Q/Q730E or 5SNP were assayed for trimming and the relative maximum B3Z response compared to WT calculated. Bars show results pooled from at least six experiments±SEM (***; P<0.001, ns; not significant). From left to right bars show results with WT, M349V, K528R, M349V/K528R, M349V/D575N/R725Q, K528R/R725Q, K528R/Q730E, R725Q/Q730E and 5SNP (B) Erap1-deficient fibroblasts were transfected with the indicated ERAP1 haplotype together with X-SHL8 minigenes representing 18 amino acids and assessed for generation of SHL8 by B3Z activation. The relative presentation of SHL8 was compared to that observed for Erap1-deficient fibroblasts transfected with WT ERAP1 and ES-M-SHL8. Dashed lines indicate 50% of ES-M-SHL8 generation. Data are pooled from three separate experiments.

FIG. 5. ERAP1 allele protein expression in Erap1-deficient transfected fibroblasts. Erap1-deficient fibroblasts were transfected with WT, E320A or ERAP1 alleles identified from samples (as indicated). After 48 hours cells were harvested and lysates from 2×10⁵ cell equivalents run on a 10% SDS-PAGE gel. The presence of ERAP1 or GAPDH (loading control) protein was detected using α-ARTS1 or α-GAPDH antibodies.

FIG. 6. B3Z T cell hybridomas do not recognize N-terminally extended SHL8 peptide. SHL8 and all N-terminally extended intermediates up to AIVMK-SHL8 (as indicated) were incubated in the presence () or absence (∘) of trypsin. After 3 hours, H-2K^{b} expressing L cells and B3Z T cell hybridoma was added and incubated overnight. B3Z stimulation was assessed using CPRG and measured at 595 nm wavelength.

FIG. 7. SNP cis interactions affect ERAP1 trimming. (A) Erap1-deficient fibroblasts were transfected with X5-SHL8 and WT, R725Q ERAP1 or R725Q containing a second disease associated SNP, M349V, K528R, D575N or Q730E and assessed for trimming using the B3Z T cell hybridoma. The relative maximum B3Z response of transfectants compared to WT was calculated. Data is pooled from four experiments±SEM (***; P<0.001, *; P<0.05). (B) Erap1-deficient fibroblasts were transfected with X5-SHL8 and WT, K528R or K528R/D575N ERAP1 and assessed for trimming using the B3Z T cell hybridoma. (C) The relative maximum B3Z response of K528R and K528R/D575N compared to WT ERAP1 was calculated. The results show data pooled from four experiments±SEM (***; P<0.001).

FIG. 8. Trimming of the N-terminally extended model antigen X5-SHL8 by SNP variant ERAP1. (A) Erap1 deficient cells were transfected with X5-SHL8 and WT, E320A, or SNP variant M349V, K528R, D575N, R725Q or Q730E ERAP1 and assayed for trimming by stimulation of the LacZ-inducible, SHL8/K^{b}-specific B3Z T cell hybridoma. Data are representative of six separate experiments. (B) Erap1 deficient cells were transfected as for A and the relative maximum B3Z response of SNP variant ERAP1 trimmed X5-SHL8 compared to WT was calculated. Bars show results pooled from six experiments±SEM (***; P<0.001, ns; not significant). (C) Erap1 deficient cells were transfected with WT, E320A or SNP mutated ERAP1 variants (as indicated). After 48 hours cells were harvested and lysates from 2×10⁵ cell equivalents run on a 10% SDS-PAGE gel. The presence of ERAP1 or GAPDH (loading control) protein was detected using α-ERAP1 or α-GAPDH antibodies.

FIG. 9. ERAP1 haplotype combinations isolated from AS cases have impaired trimming capacity. Erap1 deficient cells were transfected with ERAP1 haplotypes corresponding to individual haplotype combinations (1=WT, 2=5SNP, 3=K528R/Q730E, 4=K528R, 5=M349V/D575N/R725Q, 6=K528R/R725Q, 7=R725Q/Q730E, 8=M349V, 9=M349V/K528R) and X5-SHL8 and assessed for trimming using B3Z. (A, B) Representative line graphs showing trimming of the most common haplotype combinations from cases (A) or controls (B) as indicated. The positive (WT) and negative control (E320A) haplotypes was also transfected. Data are representative of four experiments. (C) The relative maximum B3Z response of all observed haplotype combinations identified compared to WT haplotype are shown. Bars show results pooled from four experimental repeats±SEM (***; P<0.001, ns; not significant). (D) The ability of ERAP1 haplotype combinations to restore MHC I levels in Erap1 deficient cells was assessed. Erap1 deficient cells were transfected with haplotype combinations (WT+K528R/Q730E and M349V/D575N/R725Q+K528R/Q730E not done) and the levels of H-2K^{b} (black bars) or H-2D^{b} (white bars) assessed and compared to WT. Results show data pooled from three experiments±SEM (***; P<0.001, **; P<0.01, *; P<0.05, ns; not significant). The dashed line represents 100% restoration of MHC I levels.

FIG. 10. Co-expression of ERAP1 haplotypes in Erap1 deficient cells. Erap1 deficient cells were transfected with ERAP1 haplotypes corresponding to individual haplotype combinations (1=WT, 2=5SNP, 3=K528R/Q730E, 4=K528R, 5=M349V/D575N/R725Q, 6=K528R/R725Q, 7=R725Q/Q730E, 8=M349V, 9=M349V/K528R) identified from either cases (A) or controls (B) as indicated. The combined ERAP1 haplotypes were transfected and assessed for peptide trimming using B3Z hybridoma. The positive (WT; closed circles) and negative control (E320A;open circles) haplotypes was also transfected. Data are representative of four experiments. (C) Haplotype #1 ERAP1 molecules were tagged with a C terminal V5 epitope and detected with α-V5 antibody. Haplotype #2 ERAP1 were tagged with a C terminal HA epitope and detected with α-HA antibody. Co-expression of ERAP1 molecules in transfected Erap1 deficient cells was confirmed from 2×105 cell equivalent lysates by the presence of V5 and HA tagged ERAP1 molecules. GAPDH was used as loading controls. (D and E) To preclude differential expression of individual ERAP1 haplotypes in transfected cells, reciprocal haplotype combination experiments were performed using ERAP1 haplotypes with the opposite tag. The same overall peptide trimming phenotype was observed for haplotype combinations irrespective of which tag each haplotype possessed.

FIG. 11. Phylogenetic analysis of ERAP1 allotypes. ERAP1 amino acid (A) and nucleotide (B) sequences were used to generate unrooted phylogenetic trees. The relative and absolute frequency of each allotype in the tree is indicated. The relative trimming function of each allotype is also indicated; Hyper-active trimmers are boxed, hypo-active trimmers solid underline, intermediate trimmers dashed underline, efficient trimmers bold type. Allotypes in italics have not been assessed.

FIG. 12. ERAP1 allotype pairs isolated from AS cases have impaired trimming capacity. Erap1 deficient cells were transfected with ERAP1 allotypes corresponding to individual allotype pairs identified in cases and controls and X5-SHL8 and assessed for trimming using B3Z. (A, B) Representative line graphs showing trimming of the most common allotype pairs from controls (A) or cases (B) as indicated. The positive (ERAP1*002) and negative control (ERAP1*002 E320A) allotypes were also transfected. Data are representative of four experiments. (C, D) The relative maximum B3Z response of observed allotype pairs identified in control or case groups (C) or individual allotype pairs (D) compared to ERAP1*002 allotype pair are shown. Bars show results pooled from four experimental repeats±SEM (****; P<0.0001, **; P<0.01; *; P<0.05 ns; not significant).

FIG. 13. AS case ERAP1 allotype pairs fail to increase HLA-B27 cell surface expression. Flow cytometry analysis of HLA-B27 cell surface expression by Erap1^{-/-} fibroblasts (A-C) or ERAP1 KO 293T cells (D) transfected with each of the 15 ERAP1 allotype pairs identified from control and AS case groups compared to ERAP1*002 E320A. (A, D) Representative histograms showing HLA-B27 expression after transfection of Erap1^{-/-} fibroblasts (A) and ERAP1 KO 293T cells (D) with an example of allotype pairs from control and AS case groups. (B) Comparison of HLA-B27 cell surface expression in Erap1^{-/-} fibroblasts (B) transfected with allotype pairs from control and AS case groups. Each symbol represents a single allotype pair transfection from three (B) independent experiments; ***, P<0.0001. (C) The effect of individual ERAP1 allotype pairs from control and AS cases on HLA-B27 cell surface expression following transfection into Erap1 fibroblasts (C). Data are pooled from three independent experiments±SEM (C).

FIG. 14. Model for how the ERAP1 trimming activity of an allotype pair trimming links with disease. ERAP1 allotype pairs from individuals have a broad spectrum of trimming activity. Those with trimming activities toward the extreme ends of this spectrum have a greater risk of developing AS. This increased risk is manifested in two different ways: i) Over-trimming ERAP1 activity results in increased misfolded and HLA-B27 homodimers in the ER inducing the unfolded protein response. ii) Under-trimming ERAP1 activity results in increased cell surface HLA-B27 homodimers activating NK and/or Th17 cells.

### DETAILED DESCRIPTION OF THE INVENTION

### The Disease Condition which is Diagnosed and/or Treated

The condition which is diagnosed and/or treated is one which relates to ERAP1 function, i.e. an ERAP1 associated disease. Preferably the condition is a spondylarthropathy or arthritis, such as AS, psoriatic arthritis or reactive arthritis. The condition may be psoriasis, type-1 diabetes, cervical carcinoma or head and neck squamous cell carcinoma.

### The Individual that is Typed and/or Treated

The individual is typically a human, such as from a Caucasian population, a Chinese population or an African population. The individual may be from a European population. The individual may be suspected of being at risk of the relevant condition. The individual may have one or more symptoms of the condition. However in one embodiment the individual does not have any symptoms of the disease. The individual may be at risk of the condition because of exposure to known genetic or environmental risk factors. The individual may have a parent or a sibling with the condition.

Where the disease is a spondylarthropathy (such as AS) or an arthritis, the individual may be positive for HLA B27. The individual may have back pain, and in one embodiment has had back pain for more than 1 year. The individual may be seronegative (i.e. be negative for rheumatoid factor)

### Purpose of Haplotype Detection

The haplotype detection method of the invention may be carried out to diagnose presence or susceptibility to any of the conditions mentioned herein. It may be used to diagnose the subset of disease or to provide a prognosis for the disease. Thus the method may be used to determine the likely course of the disease and, for example, how aggressive the condition is likely to be, particularly for AS. The method can be used to select an appropriate therapy type (for example which therapeutic agent should be used) or therapy schedule (for example the dosage of the therapy which is given). The method may be used to predict the response of the individual to a specific treatment. These embodiments are discussed further in sections below.

### The Haplotype which is Detected

The ERAP1 haplotype of an individual refers to the combination of SNP's present in the ERAP1 gene region which are generally inherited together in the population. The ERAP1 region includes the ERAP1 gene itself and its associated up- and down-stream regulatory regions. An ERAP1 haplotype can be defined by sets of SNP's that are inherited together in blocks. Any (such as all) of the SNP's of the haplotype may be present in the coding region. Any (such as all) of the SNP's may cause a change in the sequence of the expressed protein. The haplotype typically causes a change in the expression (i.e. amount expressed) or activity of the ERAP1 protein.

SNP's and haplotypes are defined relative to the wild type sequence. Thus when the method is being defined in terms of typing SNP's and haplotypes shown in the Tables herein it is understood that this will normally exclude typing of the wild type haplotype. The method may comprise typing any of SNP's or haplotypes shown in any of the Tables. The term ‘typing’ typically refers to determining presence or absence of the relevant SNP or haplotype.

In one embodiment at least one or more of the following haplotypes are typed R725Q/Q730E, K528R/R725Q and the 5SNP haplotype of Table III. In another embodiment at least one of the following five SNPs is typed I82V, L102I, P115L, S199F or S581L, and all four of M349V, K528R, R725Q, Q730E are typed, and optionally D575N is also typed.

The haplotype will comprise at least 2 SNP's, and thus may comprise 3, 4, 5, 6, 7 or more SNP's. The haplotype typically comprises at least 1, 2, 3, 4 or more of the SNP's shown in Table I. Preferably the haplotype is any of haplotypes 2 to 9 as defined in Table III. The haplotype may cause a hypo or a hyper trimming activity in the expressed protein. 2, 3, 4 or all of the SNP's within the haplotype may be least 20 nucleotides apart from each other.

The SNP's shown in Table VI are associated with susceptibility to disease and are found in combination with certain haplotypes as described. In one embodiment the method comprises typing any of the haplotypes 2 to 9 as shown in Table III and additionally typing any or 1, 2, 3, 4 or more of the SNP's shown in Table VI.

In one embodiment the method comprises determining whether any of the haplotypes shown in Table XIV, Table XV, Table XVI, Table XVII, XVIII, Table XIX, Table XX, Table XXI or Table XXII are present in or absent from the genome of the individual, wherein optionally the method is being carried out for diagnosis of the condition or purpose mentioned in the relevant Table.

### Detection of the Haplotype

The invention relates to typing haplotypes in ERAP1. This can be done by analysing the ERAP1 gene or a nucleic acid derived from the gene, such as mRNA or cDNA. Thus detection can be performed by genetic typing, which usually determines the identity of the nucleotide present at a defined position. The typing may be done by analysis of the ERAP1 protein. One or both alleles (chromosomes) of the individual may be typed. One or both forms of the expressed protein may be typed.

### Samples from the Individual

Detection may be carried out in vitro on a suitable sample from the individual, wherein the sample typically comprises nucleic acid and/or ERAP1 protein from the individual. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the polymorphism.

### Genetic Typing and Protein Typing

The detection or genotyping of polymorphisms may comprise contacting a polynucleotide or polypeptide of the individual with a specific binding agent for the polymorphism and determining whether the agent binds to the polynucleotide or polypeptide, wherein binding of the agent indicates the presence of the polymorphism, and lack of binding of the agent indicates the absence of the polymorphism. The method generally comprises using as many different specific binding agents as is required to ascertain the presence of the relevant haplotype(s). 1, 2, 3, 4, 5, 6 or more different specific binding agents may be used. In one embodiment a kit is provided comprising the specific binding agent(s) and then haplotype detection is carried out using the specific binding agent(s) in the kit.

A specific binding agent is an agent that binds with preferential or high affinity to the polynucleotide or polypeptide having the polymorphism but does not bind or binds with only low affinity to other polynucleotides or polypeptides. The specific binding agent may be a probe or primer. The probe may be a protein (such as an antibody) or an oligonucleotide. The probe may be labelled or may be capable of being labelled indirectly. The binding of the probe to the polynucleotide or polypeptide may be used to immobilise either the probe or the polynucleotide or protein.

Generally in the method, determination of the binding of the agent to the polymorphism can be carried out by determining the binding of the agent to the polynucleotide or polypeptide of the individual. However in one embodiment the agent is also able to bind the corresponding wild-type sequence, for example by binding the nucleotides/amino acids which flank the polymorphism position, although the manner of binding to the wild-type sequence will be detectably different.

The method may be based on an oligonucleotide ligation assay in which two oligonucleotide probes are used. These probes bind to adjacent areas on the polynucleotide which contains the polymorphism, allowing after binding the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system. In such a system when the probe is bound to polynucleotide sequence containing the polymorphism it forms a heteroduplex at the site where the polymorphism occurs and hence does not form a double strand structure. Such a heteroduplex structure can be detected by the use of single or double strand specific enzyme. Typically the probe is an RNA probe, the heteroduplex region is cleaved using RNase H and the polymorphism is detected by detecting the cleavage products.

The method may be based on fluorescent chemical cleavage mismatch analysis. In one embodiment a PCR primer is used that primes a PCR reaction only if it binds a polynucleotide containing the polymorphism, for example a sequence- or allele-specific PCR system, and the presence of the polymorphism may be determined by the detecting the PCR product. Preferably the region of the primer which is complementary to the polymorphism is at or near the 3′ end of the primer. The presence of the polymorphism may be determined using a fluorescent dye and quenching agent-based PCR assay such as the Taqman PCR detection system.

The presence of the polymorphism may be determined based on the change which the presence of the polymorphism makes to the mobility of the polynucleotide or polypeptide during gel electrophoresis. In the case of a polynucleotide single-stranded conformation polymorphism (SSCP) or denaturing gradient gel electrophoresis (DDGE) analysis may be used. The presence of the polymorphism may be detected by means of fluorescence resonance energy transfer (FRET). The polymorphism may be detected by means of a dual hybridisation probe system. In one embodiment a polymorphism (or the haplotype as a whole) is detected using a polynucleotide array, such as a gene chip.

Primers and probes which can be used in the invention will preferably be at least 10, preferably at least 15 or at least 20, or at least 40 nucleotides in length. They will typically be up to 40, 50, 60, 70, 100 or 150 nucleotides in length. They may be present in an isolated or substantially purified form. They will usually comprise sequence which is completely or partially complementary to the target sequence, and thus they will usually comprise sequence which is homologous to ERAP1 gene sequence. The skilled person will of course realise that references herein to sequences that are homologous to the ERAP1 sequences and which target (bind) ERAP1 sequences includes sequences which are complementary to homologues of ERAP1 sequences.

Polymorphisms may be detected by sequencing a region comprising the polymorphism, which may include sequencing the entire ERAP1 gene or coding sequence.

In embodiments where ERAP1 protein is typed, one or more polymorphism-specific or haplotype-specific antibodies may be used.

### Extent of Haplotype Typing

Typically in the method of the invention the presence or absence of the haplotypes mentioned in Table I is detected. In one embodiment, whether or not the genome of the individual comprises 1, 2, 3, 4, 5, 6, 7 or all of the haplotypes listed in Table I is ascertained. In one embodiment 3, 4, 5, 6 or more, or all of the nucleotide positions shown in Table I are typed. In a preferred embodiment, at least 1, 2, 3, 4 or 5 of the SNP's shown in Table I are typed.

### Typing by Measuring Activity of ERAP1

In one embodiment the activity of the ERAP1 protein is detected to ascertain the presence of a hypo or hyper haplotype. Typically this comprises detection of the aminopeptidase activity, for example by detection of trimming activity. The skilled person will be able to detect hypo or hyper activity by the means available in the art. The activity of the wild type ERAP1 protein may be used to define normal activity, and thus activities which are more or less than this can be used to define hyper and hypo activity respectively. Alternatively hypo or hyper activity can be defined using the activities of specific haplotypes disclosed herein which have hypo or hyper activities.

Trimming activity may be measured using any suitable assay. In one embodiment the ERAP1 protein is expressed in an ERAP1 deficient cell line and then expression of peptides presented on the cell surface is analysed. In one embodiment the ERAP1 protein is contacted with a suitable peptide under conditions where the wild type ERAP1 protein would trim (cut) the peptide, and whether or not trimming occurs and/or rate of trimming of the peptide is detected either by detection of the amount of the original peptide or by detection of a product of the trimming reaction.

### Detailed Description of Embodiments of the Invention

In one embodiment there is an assessment of the function of ERAP1 from individuals. A blood sample is taken and either used directly or PBMC are isolated by density centrifugation (e.g. ficoll). A cell lysate is made from the sample using NP-40 detergent cell lysis buffer and centrifugation to remove cell membranes. The supernatant is added to a well that has been pre-coated with anti-ERAP1 antibody and incubated for 1 hr. Cell lysis may be performed directly in the pre-coated wells. After the ERAP1 has bound to antibody the unbound proteins are removed by washing. The function of the ERAP1 proteins within the well are assessed by the addition of a colorimetric or fluorogenic substrate that either changes colour or fluoresces when ERAP1 has trimmed. The degree of colour change or amount of fluorescence can be used to detect the relative activity of the ERAP1 proteins. Should the antibody block ERAP1 action, ERAP1 can be disassociated from the antibody by heat or by low pH. The activity of ERAP1 can then be assessed when the temperature is reduced or the pH is neutralised.

A variation on the method could use haplotype specific anti-ERAP1 antibodies. Detection would be by standard ELISA methodology. Following binding of ERAP1 to the haplotype specific anti-ERAP1 antibody the presence of ERAP1 is detected with incubation with a second anti-ERAP1 antibody (not haplotype specific). After binding, a horseradish-peroxidase conjugated secondary antibody which is raised to the host species of the anti-ERAP1 antibody (e.g. goat anti-rabbit Ig-HRP). A colorimetric substrate of HRP is added to detect the presence of ERAP1.

### Detecting the Subset of Disease and Therapy

In one embodiment diagnosis may be carried out to detect the subset of the disease or to ascertain prognosis of the condition. This allows prediction of disease progression and outcome. It also allows appropriate selection of patient treatment. Possession of a hyper trimming haplotype is likely to result in a more aggressive disease condition and faster progression of disease. Thus detection of a hyper trimming haplotype could lead to increased dosage of a therapeutic agent being administered or selection of an agent with high activity. The method allows responsiveness to treatment to be determined, particularly in individuals who have AS. In particular it allows responsiveness to NSAIDS to be determined.

In one embodiment the invention provides a therapeutic agent for AS for use in a method of treatment of a subset of AS in an individual, wherein method comprises choosing said agent by the detection method of the invention and administering the chosen agent to the individual. The agent may be an analgesic, a non-steroidal anti-inflammatory drug, a corticosteroid or a disease modifying anti-rheumatic drug (DMARD). Therapeutic agents may be administered in association with appropriate diluents or carriers. They may be administered by appropriate routes, such as intravenously. They may be administered in appropriate amounts, such as effective, non-toxic amounts. In one embodiment the method of the invention is used to select individuals based on whether not they will respond to a particular treatment.

### A Kit for Carrying Out the Invention

A kit may be produced for carrying out the method of the invention. The kit may comprise means for determining the presence or absence of one or more polymorphisms in an individual which define the ERAP1 haplotype or disease susceptibility of the individual. In particular, such means may include a probe, primer, pair or combination of primers, or antibody, including an antibody fragment, as defined herein which is capable of detecting or aiding detection of a polymorphism. The kit typically includes a set of instructions for carrying out the method.

### Homologous Sequences

Homologous sequences are mentioned herein. Such sequences typically have at least 70% homology, preferably at least 80%, 90%, 95%, 97% or 99% homology with the original sequence, for example over a region of at least 15, 20 or 40 or more contiguous nucleic acids (of the original sequence). Methods of measuring homology are well known in the art and it will be understood by those of skill in the art that in the present context, homology is calculated on the basis of nucleic acid identity (sometimes referred to as “hard homology”).

For example the UWGCG Package provides the BESTFIT program which can be used to calculate homology (for example used on its default settings) (Devereux et al (1984) Nucleic Acids Research 12, p 3 87-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.Software for performing BLAST analyses is publicly available through the National Center for Biotechnology Information (http://www.ncbi.nlm.nih.gov/).

This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands. The BLAST algorithm performs a statistical analysis of the similarity between two sequences; see e. g., Karlin and Altschul (1993) Proc. Natl. Acad. Sci. USA 90: 5873-5787. One measure of similarity provided by the BLAST algorithm is the smallest sum probability (P (N)), which provides an indication of the probability by which a match between two nucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs from the original sequence by no more than 2, 5, 10, 15 or 20 mutations (which may be substitutions, deletions or insertions). These mutations may be measured across any of the regions mentioned above in relation to calculating homology.

The invention is illustrated by the following Examples:

### EXAMPLE 1

### Detection of Functionally Distinct Haplotypes in ERAP-1

Major Histocompatibility complex class I (MHC I) molecules display peptides of 8-10 mer amino acids in length at the cell surface for immune surveillance by circulating cytotoxic T cells (CD8+ T cells). MHC I samples the intracellular proteome and presents peptides derived from self-proteins, including those that are aberrantly expressed in cancer, as well as proteins originating from intracellular viruses and bacteria. Cytosolic proteases, including the proteasome, generate peptides with a precise C terminus but a mixture of N-terminally extended intermediates, which are then transported into the endoplasmic reticulum (ER) by the transporter associated with antigen processing (TAP). Here, further processing in the form of N-terminal peptide trimming by ERAP1 can occur, with the net result of increasing the frequency of peptides that are of an appropriate length to bind to MHC I. Some antigenic peptides can be destroyed or “over-processed” by ERAP1, indicating that ERAP1 has a role as an antigenic peptide editor, influencing the peptide repertoire displayed at the cell surface. In humans, ERAP2, a homologue of ERAP1 is also able to perform this function.

The ability of ERAP1 to trim N-terminal amino acids from epitope precursors has been shown to depend on the amino acids present, which are removed at vastly different rates, forming a distinct hierarchy. This specificity ultimately defines the abundance of presented peptide antigens which in turn can shape the immunodominance of CD8+ T cell responses to pathogens and cancer. Recent genome wide association studies (GWAS) have identified polymorphisms encoded within ERAP1 linked to many diseases such as cervical carcinoma and the autoimmune diseases, ankylosing spondylitis (AS), multiple sclerosis and psoriasis. Individual amino acid changes within ERAP1, corresponding to the SNPs, and their effect on peptide trimming activity has been investigated. These studies did not examine the effect of multiple SNPs/haplotypes on the ability of ERAP1 to trim peptide precursors or their effects on amino acid specificity. Genetic studies on AS have investigated ERAP1 SNP haplotypes (K528/D575/R725), (K528/D575/Q730E) and ERAP1/ERAP2 haplotypes (Q730/K528 ERAP1 and K392N ERAP2). These studies examined haplotypes containing only certain SNPs identified in the original GWAS study and did not examine their function.

Therefore, the extent to which SNPs assemble into haplotypes is not known, nor whether the ERAP1 alleles encoded by the different ERAP1 haplotypes might have different functions. We have identified nine naturally occurring ERAP1 haplotypes from individuals, based on the five disease associated SNPs. The ERAP1 alleles encoded by these haplotypes displayed three generic activities (efficient, hypo- and hyper-functional) based on the precise substrate specificity of each allele highlighting the importance of ERAP1 alleles in the generation of the peptide repertoire.

Materials and Methods

Subjects. Samples were recruited from the Department of Rheumatology, University Hospital Southampton NHS Foundation Trust and obtained in the Southampton National Institute for Health Research Wellcome Trust Clinical Research Facility, University Hospital Southampton NHS Foundation.

ERAP1 isolation and generation of ERAP1 sequence variant E320A. RNA purified from 2×10⁶ CEM (human T cell lymphoblast-like cell line) cells with RNeasy mini kit (Qiagen) or 200 μl blood with ZR whole-blood RNA prep (Zymo Research) was used to generate cDNA with the Transcriptor High Fidelity cDNA synthesis kit (Roche). ERAP1 was amplified from cDNA using KOD Hot Start DNA polymerase (Merck) and the following primers:

5′ primer (EcoRI site in italics),
5′-GACGAATTCATGGTGTTTCTGCCCCTCAAATG-3′;
3′ primer
5′-GACCTCGAGCATACGTTCAAGCTTTTCAC-3′ (Sigma).

(XhoI site in italics),

The PCR amplification product was cloned into the vector pcDNA3.1 (Life Technologies). Site directed mutagenesis (SDM) was used to generate the ERAP1 E320A non-functional variant using the WT cloned ERAP1 vector construct with KOD Hot Start DNA polymerase and the following primers (mutated nucleotide in italics): E320A

5′-CTGGTGCTATGGCAAACTGGGGACTG-3′
and
5′-CAGTCCCCAGTTTGCCATAGCACCAG-3′.

DNA constructs. The ES-SHL8, ES-X5-SHL8 and ES-X6-SHL8 DNA constructs all encode the ER targeting signal sequence and have been described previously (1, 2). ES-X-SHL8 constructs were generated by the incorporation of an additional amino acid into the ES-SHL8 construct using the following primers: 5′-GCAGTCTGCAGCGCGNNSAGCATCATCAACTTCG-3′ and 5′-CGAAGTTGATGATGCTSNNCGCGCTGCAGACTGC-3′ where N=any nucleotide and S=C or G, resulting in amino acids being represented. Constructs were sequence verified and the most frequent codon for each amino acid chosen for use where possible.

Cell lines, transfection and T cell activation assays. An Erap1-deficient fibroblast cell line used for all transfection experiments was cultured as described previously (I). Culture conditions for B3Z T cell hybridoma and H-2K^{b}-L cells have been described before (2). Erap1-deficient fibroblasts were transfected with 1 μg of each ERAP1 haplotype and ES-AIVMK-SHL8 (X5-SHL8) or ES-LEQLEK-SHL8 (X6-SHL8) minigene construct (3) (pcDNA3.1) or SCT using FuGENE 6 (Roche). Where N-terminal amino acid specificity was assessed, 0.05 μg of each ERAP1 haplotype and 0.05 μg of each X-SHL8 minigene construct were transfected together in a 96 well plate. After 48 hours, cells were harvested and incubated overnight with the LacZ inducible B3Z T cell hybridoma, specific for the recognition of SHL8/H-2K^{b} complexes at the cell surface.

Intracellular LacZ was measured with the substrate chlorophenolred-β-D-galacto-pyrannoside (Roche) by its absorbance at 595 nm and 655 nm as reference.

Single chain trimer constructs. H-2K^{b}/SL8 disulfide trap single chain trimer construct was cloned into pcDNA3.1 with EcoRV and NotI. A lysine residue preceding SL8 was added by PCR using the following primers: (lysine is in italics) 5′-GACCGGTTTGTATGCTAAAAGTATCATTAATTTCG-3′ and 5′-CGAAATTAATGATACTTTTAGCATACAAACCGGTC-3′. SDM of lysine to histidine within SL8 and glycine to lysine within the linker between peptide and β₂M was performed using the following primers: (mutated nucleotides in italics) K-H,

5′-CTATCATTAATTTCGAACATCTTAAATGCGGTGCTAGC-3′
and
5′-GCTAGCACCGCATTTAAGATGTTCGAAATTAATGATAG-3′;
G-K,
5′-CATTAATTTCGAACATCTTAAATGCGGTGCTAGCGGTGG-3′
and
5′-CCACCGCTAGCACCGCATTTAAGATGTTCGAAATTAATG-3′.

Peptide extracts, HPLC and MS analysis. Peptides of various sequences were synthesized (GL Biochem) and their structures confirmed by mass spectrometry. Endogenous peptides were extracted from transfected Erap1-deficient fibroblasts after 48 hours. Transfected Erap1-deficient fibroblasts were lysed in 10% formic acid supplemented with 10 μM irrelevant peptide, boiled for 10 min and passed through a 10 KDa filter (Millipore). The filtrate was then fractionated by RP-HPLC (Shimadzu) on a 2.1 mm×250 mm C18 column (Vydac) over a gradient of 15-40% acetonitrile. Flow rate was maintained at 0.25 ml/min and 150 μl fractions collected in 96-well plates and dried. Trypsin (50 μg/ml; Sigma) was added to fractions to release SHL8 from N-terminally extended precursors and analyzed with B3Z T cell hybridoma and H-2K^{b}-L cells as APCs. For SCT experiments carboxypeptidase B (1 U/ml; Merck) was added to fractions following RP-HPLC fractionation to remove lysine from the peptide C-terminus. For peptide mass analysis, peptide extracts or elutions were fractionated by RP-HPLC as above and detected by mass spectrometry (Shimadzu). The presence of SHL8K (m/z=1100) and IHL7K (m/z=1013) peptides was determined using LC solutions software (Shimadzu). Synthetic peptides and buffer only runs were analyzed in identical conditions to establish retention times and the absence of sample cross-contamination.

Immunoprecipitation and immunoblots. Expression of ERAP1 was determined by immunoblot. Erap1-deficient transfected fibroblasts were lysed in 0.5% Nonidet P-40, 150 mM NaCl, 5 mM EDTA and 20 mM Tris pH7.4 supplemented with phenylmethylsulfonyl fluoride and iodoacetamide (Sigma). Proteins were separated by 10% SDS-PAGE and transferred to a nitrocellulose membrane (GE healthcare). Immunoblots were probed with anti-human ARTS1 (R&D Systems) or anti-glyceraldehyde 3-phosphate dehydrogenase (Abeam) antibodies followed by HRP-conjugated secondary antibody and SuperSignal West Pico or Femto chemiluminescent substrate (Thermo Scientific). For immunoprecipitation, lysates (10⁷ cell equivalents) were incubated with anti-H-2K^{b} antibody Y3 immobilized on protein G Dynabeads (10 μg antibody/5 mg beads; Life technologies). The beads were washed and dynabead bound SCT were incubated with trypsin (50 μg/ml) for 3 hours at 37° C. Dynabeads were removed and the supernatant collected and analyzed by western blot or HPLC/MS.

Statistical Analysis. One-way ANOVA with Dunnett's post-test was performed for analysis of differences between multiple groups and control (GraphPad prism, www.graphpad.com.).

Results

### ERAP1 Haplotypes have Different Trimming Activities

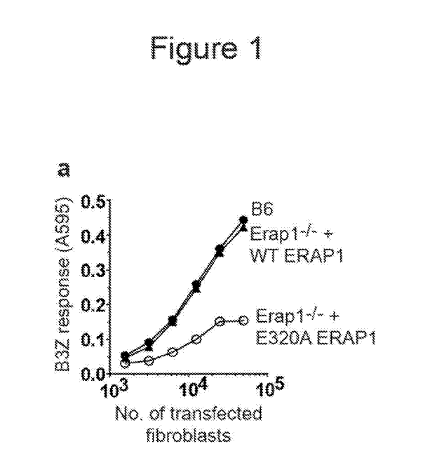

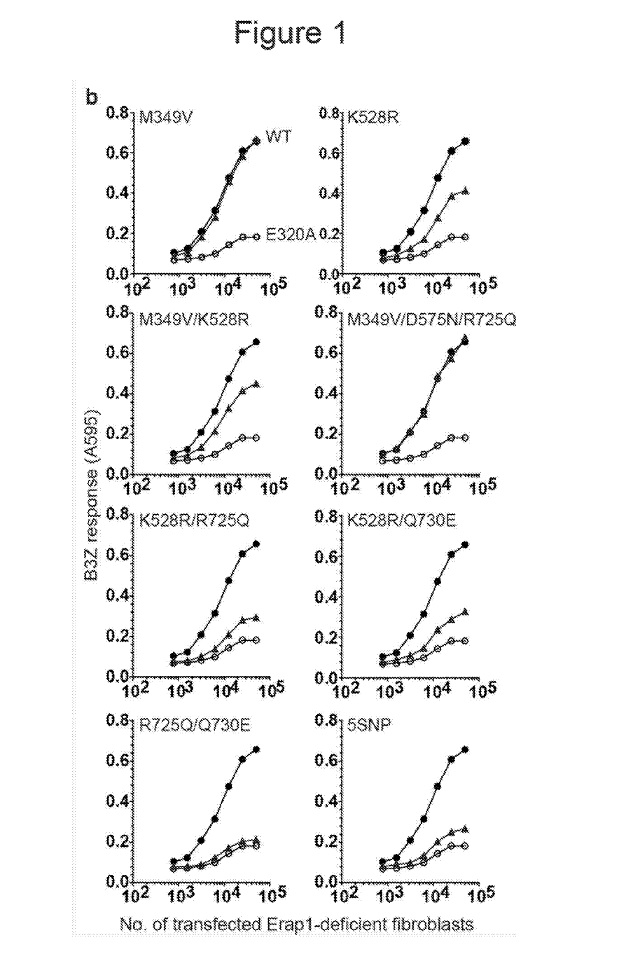

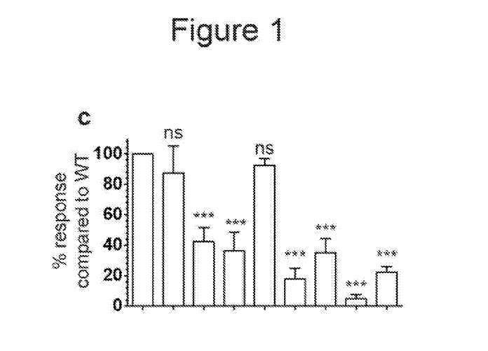

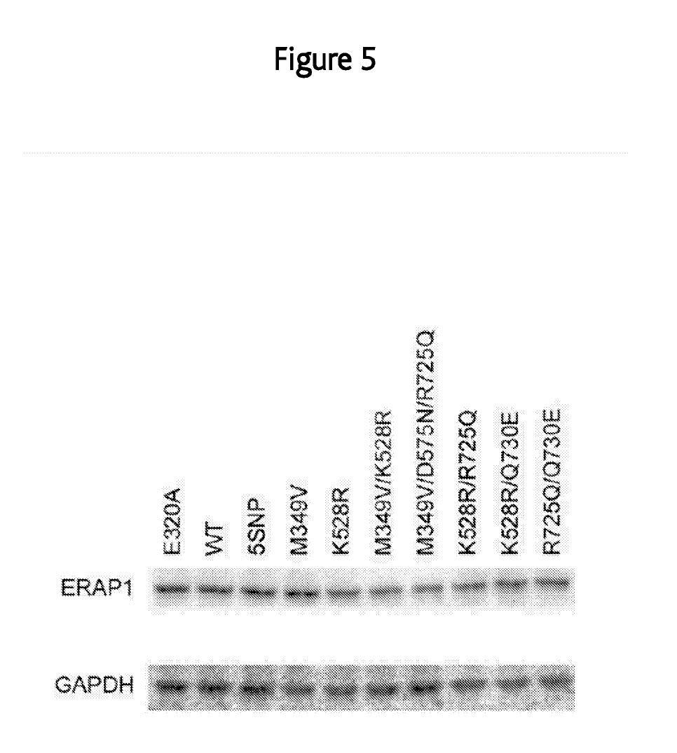

In order to determine the impact on trimming function of SNPs within ERAP1 in the context of naturally occurring haplotypes, we used molecular cloning to isolate and sequence ERAP1 genes from 20 individuals. This revealed a diverse array of ERAP1 haplotypes, mostly comprised of multiple SNP combinations based on the five SNPs with strongest disease association (Table I). The most common ERAP1 haplotype observed (cloned from CEM cells and volunteers) was identical to the previously characterized ERAP1 gene (NM 001198541.1) and termed wild-type (WT) ERAP1. To assess the trimming function of these haplotypes, we used the well characterized SIINFEHL (SHL8) murine model system in which an ER targeted (using an ER translocation signal) five amino acid N-terminally extended precursor AIVMK-SIINFEHL (X5-SHL8) was transfected into Erap1 deficient cells along with the ERAP1 haplotypes (4). The expression of trimmed SHL8 presented by H-2K^{b} at the cell surface was measured by coculturing transfected cells with the SHL8-specific T cell hybridoma B3Z, allowing a direct assessment of the trimming activity of ERAP1 haplotypes. Trimming activity in Erap1-deficient cells was <10% of that seen in WT cells following transfection with X5-SHL8 (FIG. 1A). Transfection of the WT ERAP1 sequence restored trimming activity to a level comparable to WT cells (FIG. 1A). As a negative experimental control the active site GAMEN motif, responsible for N-terminal recognition of peptide substrate, was mutated (E320A) to produce a non-functional variant (FIGS. 1A and B). Some residual trimming and SHL8 presentation is observed in Erap1-deficient cells, its source is unclear but may be from aberrant signal peptidase cleavage or an ERAP1-independent pathway. We transfected Erap1-deficient cells with X5-SHL8 and ERAP1 haplotypes and confirmed expression by western blot (FIG. 5). FIGS. 1B and C shows that two haplotypes M349V and M349V/D575N/R725Q were able to trim X5-SHL8 as efficiently as WT, with other haplotypes showing a reduced capacity. In particular, the 5SNP, R725Q/Q730E and K528R/R725Q haplotypes were least able to generate the epitope, with all three showing ≦30% of WT activity (FIGS. 1B and C).

### Functional Classes of ERAP1

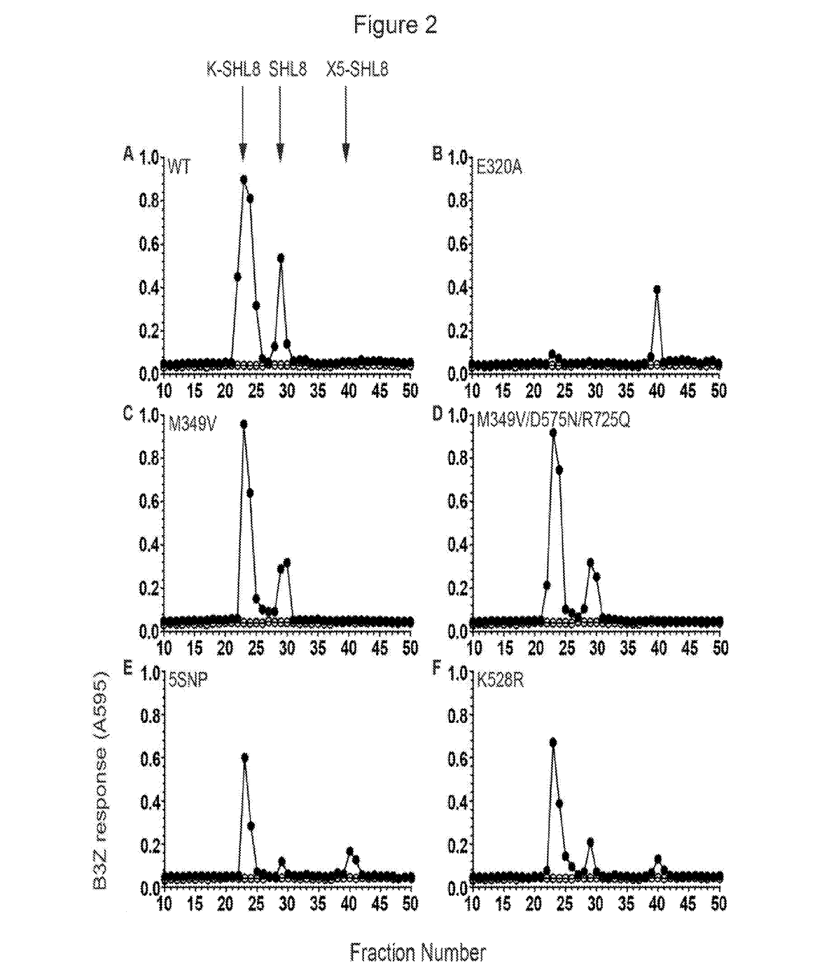

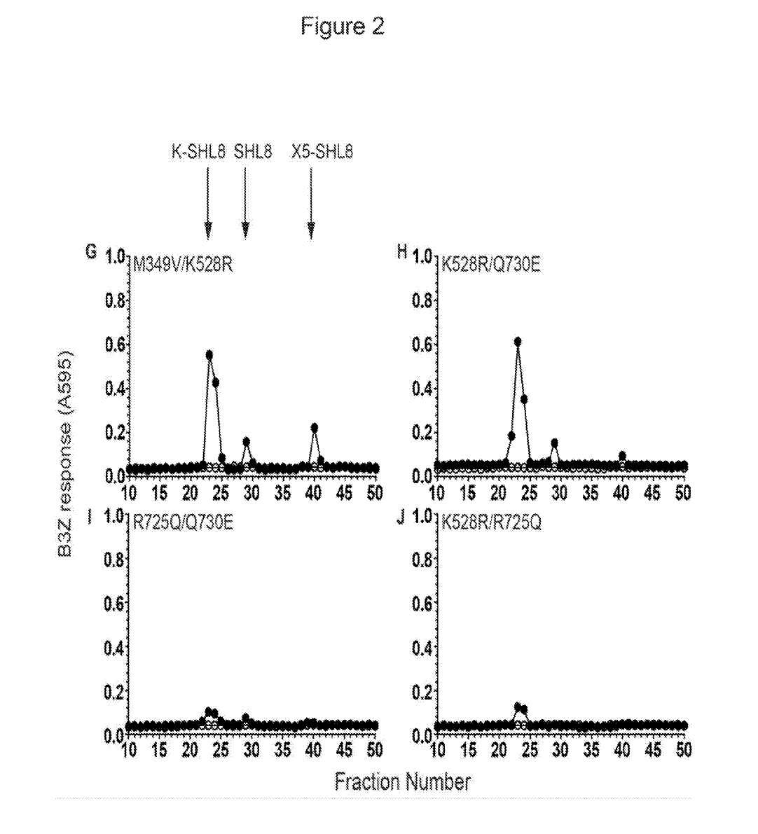

To investigate the poor trimming phenotypes and directly assess the fate of the antigenic precursors in cells, we analyzed peptide extracts by reverse-phase HPLC (FIG. 2). This method has been shown to reveal trimming intermediates at steady state, thus allowing us to identify the stage during sequential N-terminal trimming that was most affected by the polymorphisms. In Erap1-deficient cells transfected with X5-SHL8 and WT ERAP1, two peptide peaks were identified following RP-HPLC fractionation, corresponding to K-SHL8 (fraction 23) and SHL8 (fraction 29) (FIG. 2A). The peptide peak at fraction 23 originates from the capture of the N-terminal peptide trimming intermediate K-SHL8 by H-2D^{b} (5). Conveniently, this allowed us to determine the relative efficiency of the cleavage of K-SHL8 to SHL8 by ERAP1 variants by assuming that more K-SHL8 would be captured by H-2D^{b} when the K-SHL8 to SHL8 cleavage was less efficient. By contrast, when cells were transfected with E320A ERAP1 only a single peak corresponding to untrimmed precursor X5-SHL8 at fraction 40 was observed (FIG. 2B); confirming loss of function as a result of the active-site mutation. The haplotypes M349V and M349V/D575N/R725Q ERAP1 revealed peptide profiles consistent with trimming activity similar to WT (FIGS. 2C and 2D). Analysis of 5SNP, K528R, M349V/K528R and K528R/Q730E ERAP1 revealed three peaks corresponding to untrimmed X5-SHL8, K-SHL8 and SHL8 indicating a reduced ability to trim precursor peptide (FIGS. 2E-H). In all cases the ratio of K-SHL8 to SHL8 was greater than for WT (7.6, 5.4, 6.8 and 7.5 respectively compared to 4.4), consistent with their reduced ability to trim peptide precursors and indicating an inability to efficiently trim the final lysine residue. Analysis of R725Q/Q730E and K528R/R725Q ERAP1 revealed a different pattern altogether, characterized by very small K-SHL8 and SHL8 activity peaks representing <5% of the amount of trimmed product seen with WT (FIGS. 2I and 2J). The absence of additional peaks corresponding to untrimmed (X5-SHL8) or partially trimmed peptides is consistent with a hyperactive function for these variants. We therefore conclude that ERAP1 haplotypes can be grouped into three functional classes; i) efficient, ii) hypo or iii) hyper-active trimmers.

### Hyper-Functional ERAP1

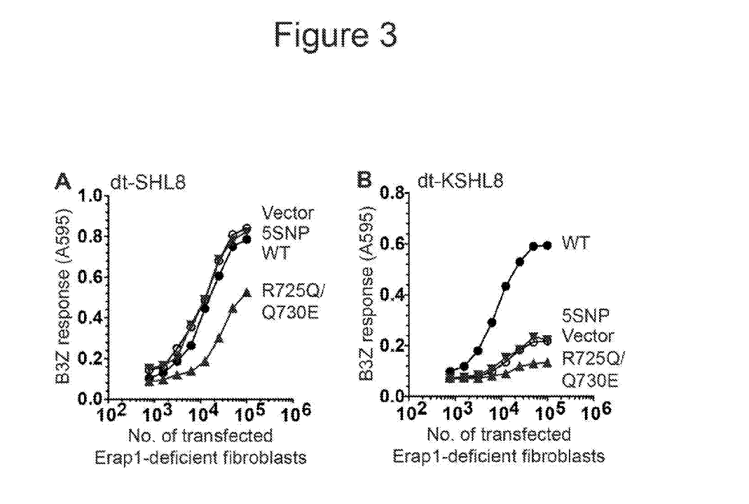

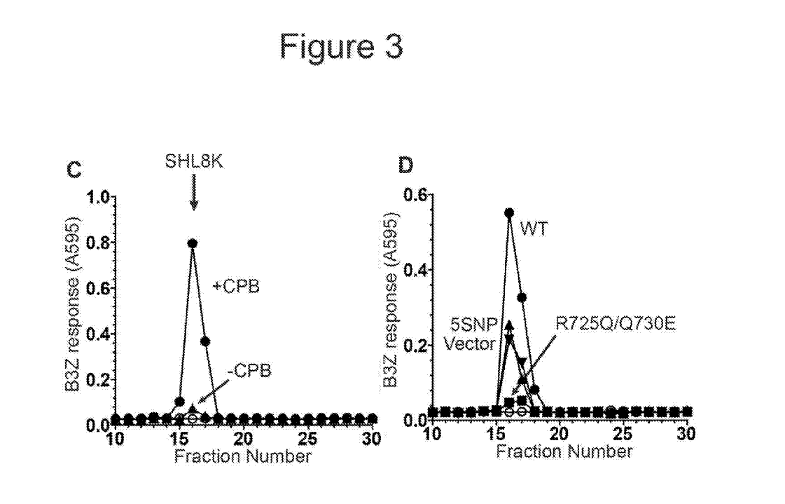

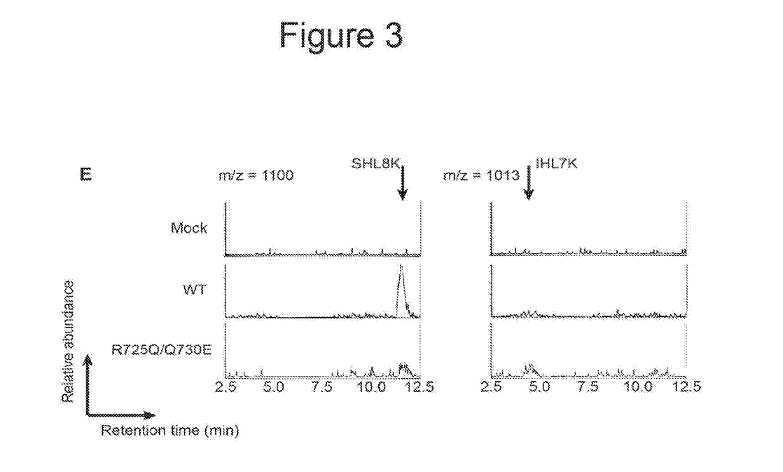

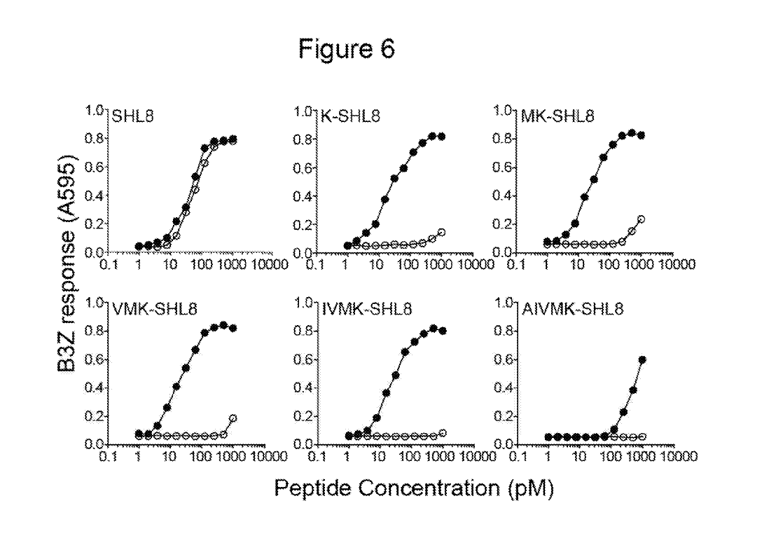

To test the hypothesis that R725Q/Q730E ERAP1 “over-trims” peptide precursors we utilized a disulfide trap single-chain MEW I construct, dt-SCT. This consists of peptide linked with β-2-microglobulin (β₂M) and MHC heavy chain in which the peptide is further tethered at its C-terminus to the MEW binding groove by introducing a disulfide bond between Y84C and a second cysteine within the peptide-β₂M linker (6). We transfected a construct containing SIINFEHL (SHL8) peptide, dt-SHL8 into Erap1-deficient cells, which was presented at the cell surface and stimulated B3Z T cells (FIG. 3A). When WT or 5SNP ERAP1 is co-expressed in these cells B3Z stimulation was unchanged. By contrast, R725Q/Q730E transfection resulted in a 50% decrease in B3Z stimulation suggesting SHL8 destruction by over-trimming (FIG. 3A). To further investigate this we generated a dt-SCT with an N-terminal extension (dt-KSHL8) which would require trimming in order for it to be presented to B3Z T cells. Disulfide trap-KSHL8 expressing Erap1-deficient cells stimulated B3Z poorly (FIG. 3B) consistent with the inability of B3Z to recognize N-terminally extended peptides (FIG. 6). When WT ERAP1 was co-expressed B3Z stimulation increased (FIG. 3B), suggesting that WT ERAP1 is able to trim the N-terminal extension from the tethered KSHL8. Co-expression of 5SNP ERAP1 did not alter B3Z stimulation compared to vector, confirming its hypo-functionality. Transfection of R725Q/Q730E in dt-KSHL8 expressing cells led to a 70% reduction in B3Z stimulation compared to vector indicating barely detectable levels of optimally trimmed peptide (FIG. 3B). This is consistent with destruction of the SHL8 epitope moiety within the dt-KSHL8 construct by overtrimming.

To gather more direct evidence for hypo and hyperfunctionality among ERAP1 variants, we introduced a trypsin cleavage site one amino acid downstream of the authentic C-terminus of SHL8 in the dt-SCT by substitution of lysine for glycine within the peptide-β₂M linker. This allowed us to recover peptide from the SCT using trypsin following immunopurification from cells. Disulfide trap-KSHL8K molecules were transfected into Erap1-deficient cells, immunopurified and eluted peptides fractionated by RP-HPLC. Fractions were treated with carboxypeptidase B to remove the C terminal lysine revealing a single peak of B3Z activity corresponding to SHL8K (fraction 16; FIG. 3C). When WT ERAP1 was transfected into dt-KSHL8K expressing cells the amount of SHL8K observed was 3-fold greater than vector alone (FIG. 3D). Transfection of 5SNP ERAP1 showed an equivalent amount of SHL8K recovered compared to vector only, thus confirming the hypoactivity of 5SNP to trim peptide precursor. The use of trypsin prior to fractionation means we are unable to recover KSHL8K which, based on the results shown in FIG. 2, we would expect to be elevated. When R725Q/Q730E was transfected, the amount of SHL8K observed was significantly reduced (>80% reduction; FIG. 3D) providing further evidence that R725Q/Q730E ERAP1 was over trimming the peptide precursor. We used mass spectrometry to identify peptide species eluted from the dt-KSHL8K molecules following RP-HPLC fractionation. A peak corresponding to the mass of SHL8K was the major species eluted from WT transfected cells, and was greatly reduced in the R725Q/Q730E transfectants (FIG. 3E). Further analysis of eluted peptides revealed a peak corresponding to IHL7K (IINFEHLK) as a unique product in the R725Q/Q730E transfectants (FIG. 3E) confirming the over-trimming function of this variant.

### Amino Acid Specificity of Defined ERAP1 Haplotypes

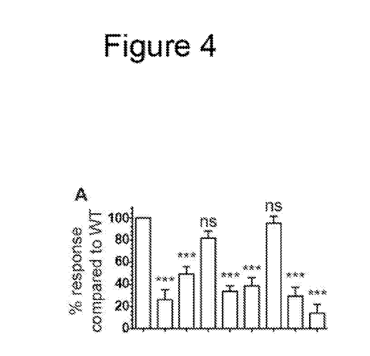

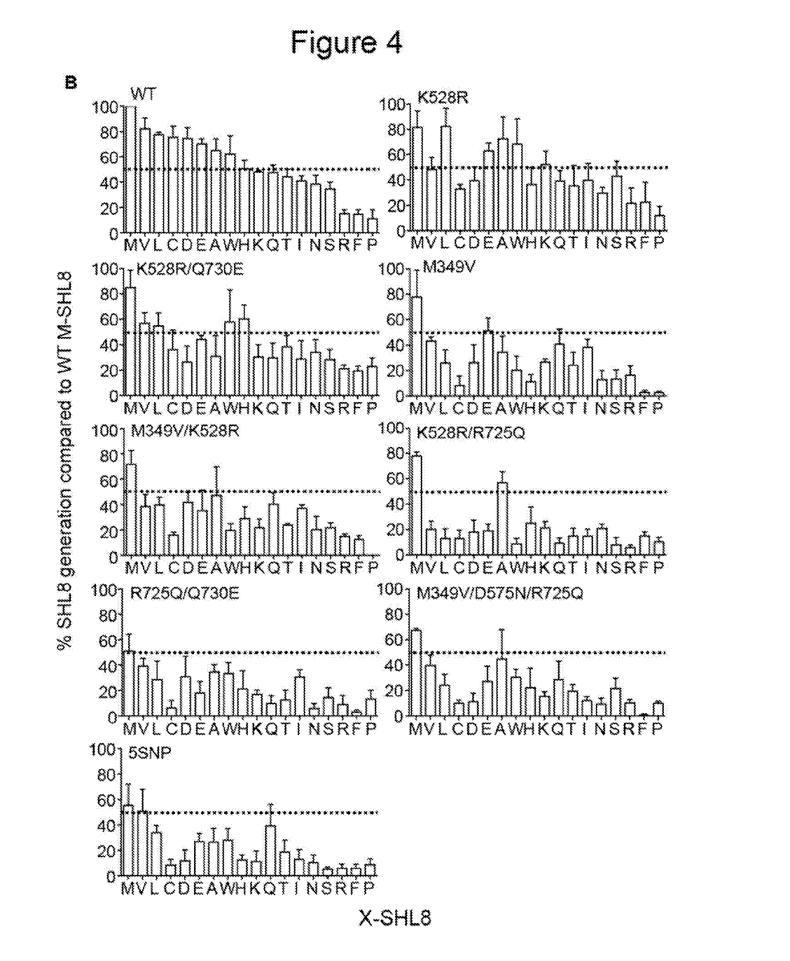

To examine whether the ability of haplotypes to generate SHL8 was dependent on the sequence of the N-terminal precursor, we substituted AIVMK for LEQLEK (X6-SHL8) containing one additional amino acid and consisting of mostly polar/charged amino acids compared to the mostly hydrophobic AIVMK extension. FIG. 4A shows that, as for X5-SHL8, most of the haplotypes showed a reduced ability to generate the final SHL8 epitope from X6-SHL8 compared to WT. Interestingly, however, M349V/K528R and K528R/Q730E, which were poor trimmers of X5-SHL8 (˜40% activity of WT), were able to efficiently process X6-SHL8. Conversely, M349V and M349V/D575N/R725Q, which trimmed X5-SHL8 well, showed a significant reduction in X6-SHL8 trimming (<40% of WT activity). This demonstrated that the activity of these haplotypes was dependent on substrate sequence, and prompted us to investigate substrate specificity in more detail.

To fine map amino acid trimming by haplotypes we utilized the ER targeted SHL8 peptide with a single amino acid extension representing 18 of the 20 amino acids (X-SHL8) transfected together with each ERAP1 haplotype. When we assessed the efficiency of SHL8 generation from each X-SHL8 substrate, we identified haplotype-specific signatures that could be broadly divided into three groups, shown in Table II and FIG. 4B: i) K528R/R725Q, R725Q/Q730E, 5SNP and M349V/D575N/R725Q were unable to generate SHL8 from the majority of amino acid precursors, ii) M349V and M349V/K528R were intermediate and could generate SHL8 from some precursors well (>75% of WT activity) and others poorly (<50% of WT activity), and iii) K528R and K528R/Q730E which, like WT, generated SHL8 well from most precursors. It is important to emphasize that this assay is not able to determine whether the lack of SHL8 presentation was due to an excess or an absence of trimming. However, it is notable that the haplotypes R725Q/Q730E, which we have shown to over-trim K-SHL8 (FIG. 3), and K528R/R725Q, which from cell surface and RP-HPLC analysis also exhibits an over-trimming phenotype, were found to have the lowest ability to generate SHL8 from all X-SHL8 substrates (Table II), suggesting that these haplotypes over-trim the majority of amino acid precursors. Interestingly, for both of these haplotypes, there are substrates where SHL8 is generated at similar levels to WT (Met and Ala for K528R/Q730E; Ile for R725Q/Q730E). This suggests that a given haplotype may have a range of activities for different N-terminal amino acids such that some are rapidly hydrolyzed and others slowly. Examination of the contribution of individual SNPs to SHL8 outcomes showed that the SNP with the strongest effect was R725Q. All haplotypes that contain this SNP (K528R/R725Q, R725Q/Q730E, M349V/D575N/R725Q, 5SNP) showed poor trimming phenotypes when assayed across the whole range of X-SHL8 substrates, and no other SNP was uniquely associated with poor trimming phenotype.

Discussion

Using molecular cloning, we identified nine discrete ERAP1 haplotypes based on the five disease associated SNPs. This confirms the polymorphic nature of ERAP1 and suggests that different haplotypes may have a role in the pathology of linked disease. Imputation and permutation haplotype studies have shown an association of ERAP1 and ERAP1/2 haplotypes with AS. Interestingly, although these studies did not examine all five SNPs examined here, the AS associated ERAP1 haplotypes (K528/D575/R725), (K528/D575/Q730E) and (Q730/K528 ERAP1 and K392N ERAP2) are represented in the haplotypes we observe, albeit being represented by more than one observed haplotype in some instances: i) K528/D575/R725=WT or M349V; ii) K528/D575/Q730E=R725Q/Q730E; iii) Q730/K528=WT, M349V or M349V/D575N/R725Q). Thus highlighting the importance of sequencing haplotypes to identify polymorphic variants. Examination of the trimming of model N-terminally extended substrates, X5- and X6-SHL8, revealed differences between haplotypes and their ability to generate SHL8, in a substrate-dependent way. Mapping of N-terminal amino acid trimming by ERAP1 haplotypes revealed a complex picture with a range of trimming abilities found among haplotypes for a given substrate and within haplotypes for a range of substrates. WT ERAP1 was found to have the greatest capacity to generate SHL8 from N-terminally extended precursors with the hierarchy of amino acid specificity showing a similar profile to those identified in previous studies using recombinant enzyme and in living cells, with any differences most likely reflecting the particular assay of choice (living cells versus recombinant enzymes and microsomal extracts). It is worth noting also that the results of previous trimming assays using transfected HeLa cells may be confounded by endogenous ERAP1 haplotypes (WT and K528R/Q730E).

Further analysis of precursor specificity shows that most variation in the generation of SHL8 between ERAP1 haplotypes is observed with substrates containing N-terminal Cys, His, Trp, Asn or Asp showing these amino acids to be the most sensitive to allelic variation in ERAP1. Analysis of N-terminal amino acid trimming specificity across haplotypes shows the amino acids Met, Val and Ala are good substrates for SHL8 generation for all haplotypes. By contrast, Arg, Pro and Phe were poor substrates with very little SHL8 generated from these precursors. Interestingly, amino acids, Cys and Asp, were only generated well by WT ERAP1 with poor generation by all other haplotypes. These analyses show that the chemical property of an amino acid does not determine whether it is a good substrate or not, however in general, hydrophobic residues are hydrolyzed more efficiently.

Comparison of haplotype trimming profiles indicated that a range of N-terminal amino acid trimming activities may exist within individual haplotypes. With an array of trimming activities (some trimmed rapidly, others slowly), those haplotypes with activities skewed to being fast are therefore likely to over-trim whereas those skewed to being slow are likely to under-trim. This observed range in ERAP1 haplotype trimming activities may reflect an evolutionary process driving trimming diversity, ensuring optimal peptide epitope generation within the population to combat disease; a similar mechanism is evident for the diversity of MHC I molecules. Therefore the more extreme phenotypes we have identified, such as hypo and hyper-active trimmers, may more commonly be found with haplotypes that trim well in the population. Instances where aberrant trimming haplotypes co-exist in an individual may therefore predispose them to disease. Our data supports the notion of AS associated haplotypes which encode ERAP1 alleles with poor trimming functions (R725Q/Q730E, M349V or M349V/D575N/R725Q and M349V; although the latter two may also encode the efficient WT ERAP1 allele), indicating a link between poor ERAP1 function and disease.

Recent crystal structures reveal an interesting link between SNPs and their effects on trimming capacity. M349V is located within the active site and although it is unlikely to directly interact with the peptide substrate, the amino acid substitution may alter the ability to form the correct catalytic conformation. Alleles which contain M349V trim amino acids poorly indicating a key role in active site maintenance. Both K528R and D575N are situated at domain junctions important for the conformation changes required for peptide trimming to occur. Similarly to M349V, alleles containing D575N have poor trimming functions, indicating its significance in allowing ERAP1 to adopt the correct conformation for trimming. By comparison, the K528R allele has an intermediate trimming phenotype suggesting a lesser role for K528R, although, like D575N, when K528R is present in multiple SNP alleles the trimming phenotype is also poor. Despite good structural data for ERAP1 very little is understood about its mechanism of action. In particular, it is not known whether the regulatory domain of ERAP1 (which contains the R725 and Q730 residues), or the MHC I peptide binding groove acts as the “molecular ruler”; extracting peptides from an iterative cycle of hydrolysis when the appropriate length is reached. Relevant to this is our observation that ERAP1 trimmed the single-chain construct efficiently despite the lack of a free C-terminus, and the strong likelihood that the C-terminal amino acid of the peptide-substrate was bound tightly in the peptide-binding groove of MHC I. This does not support a model involving an interaction between ERAP1 and the free C-terminus of peptide substrate. Trimming of small substrates such as dipeptides (unable to engage the peptide binding pocket of ERAP1) has been shown, indicating that engagement of the peptide binding pocket is not essential for trimming to occur. The ability of ERAP1 to trim the tethered peptides is most likely dependent on access to the N-terminus and related to MHC I affinity. This may therefore reflect a balance between ERAP1 and MHC I for peptide binding based on affinities. For an epitope of the correct length for MHC I binding (8-10 mer) the affinity is greater for MHC I than ERAP1 binding and therefore no further trimming occurs. However, an N-terminally extended peptide would have lower affinity for MHC I and allow binding to ERAP1, a mechanism similar to the model described by Kanaseki et at (4). The dt-SCT-SL8 system does not reflect the normal situation in the ER, but the identification of over-trimming in a system which should minimize the ability of ERAP1 to access peptides provides an alternative mechanism for ERAP1 trimming. The finding that R725Q/Q730E over-trims peptides tethered to MHC I suggests that SNPs may increase ERAP1 affinity for peptides allowing further trimming of cognate epitopes thus destroying them. It is worth noting that R725Q, which had the strongest negative effect on trimming and was uniquely included in all the haplotypes that were poor at generating SHL8 from all X-SHL8 substrates, is located within the regulatory domain of ERAP1 which has been proposed to interact with the peptide substrate.

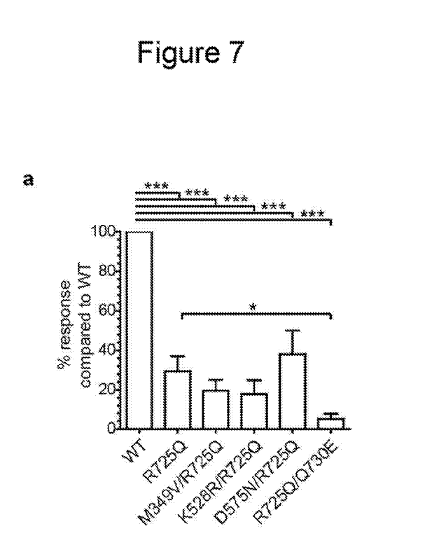

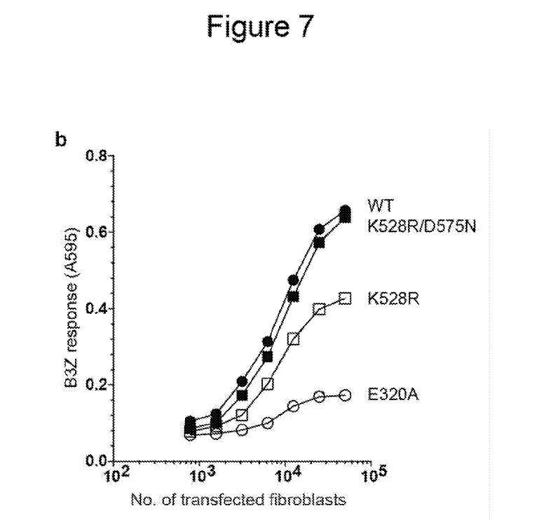

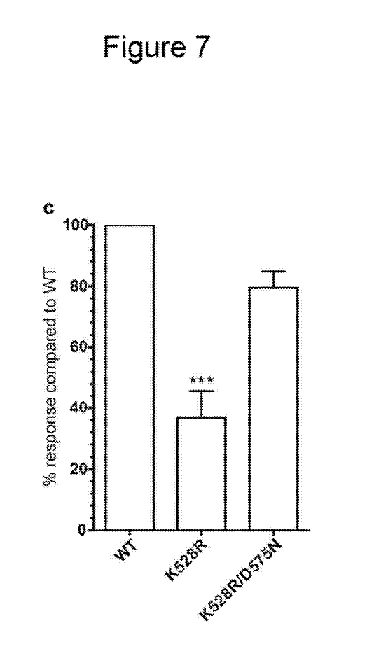

The role of disease associated SNPs on ERAP1 function has been investigated previously; single SNPs have been found to reduce trimming activity for K528R, R725Q and Q730E, but no study has investigated their affect within naturally occurring haplotypes. We have found that SNPs do not act independently and that their effect on ERAP1 function when assessed individually is not an accurate predictor of their effect when in the context of a naturally occurring haplotype. For example we found that, when assayed on X5-SHL8, a modest reduction in function seen for R725Q was amplified when additional M349V, K528R or Q730E substitutions were introduced; and although the K528R change alone reduces activity by 50%, in combination with D575N, it generates a haplotype (albeit one which we have not observed in our sample of 20 genomes) with activity close to WT (FIG. 7). Accordingly, disease association is likely to be considerably stronger when analyzed at the level of ERAP1 function than at the level of SNPs. This study indicates how epitope presentation might be influenced by ERAP1 genotype and thus impact on CTL and NK cell function.

### EXAMPLE 2

### ERAP1 Haplotypes and Distinguishing Individuals with Disease

Materials and Methods

AS cases and control samples. All samples were obtained in the Southampton National Institute for Health Research Wellcome Trust Clinical Research Facility, University Hospital Southampton NHS Foundation Trust. Diagnosis of AS was confirmed using the Assessment of SpondyloArthritis international Society (ASAS) classification criteria for axial spondyloarthritis and the modified New York criteria for the diagnosis of AS. The patient characteristics are shown in Table V.

ERAP1 isolation. RNA purified from blood (ZR RNA prep, Zymo Research) was used to generate cDNA with the Transcriptor High Fidelity cDNA synthesis kit (Roche). ERAP1 was amplified from cDNA using KOD Hot Start DNA polymerase (Merck) and the following primers: 5′ primer (EcoRI site in italics), 5′-GACGAATTCATGGTGTTTCTGCCCCTCAAATG-3′; 3′ primer (XhoI site in italics), 5′-GACCTCGAGCATACGTTCAAGCTTTTCAC-3′ (Sigma). The PCR amplicon was cloned into vectors pcDNA3.1, pcDNA3.1V5/His (Life Technologies). In addition, a modified pcDNA3.1V5/His vector substituting the V5/His for HA tag was used. Site directed mutagenesis was used to generate the ERAP1 polymorphic variants identified from the GWAS studies using the wild-type (WT) cloned ERAP1 vector constructs with KOD Hot Start DNA polymerase and the following primers (mutated nucleotide in italics): E320A

5′-CTGGTGCTATGGCAAACTGGGGACTG-3′
and
5′-CAGTCCCCAGTTTGCCATAGCACCAG-3′;
M349V
5′-CTTGGCATCACAGTGACTGTGG-3′
and
5′-CCACAGTCACTGTGATGCCAAG-3′;
K528R
5′-GGACACTGCAGAGGGGCTTTCCTCTG-3′
and
5′-CAGAGGAAAGCCCCTCTGCAGTGTCC-3′,
D575N
5′-CAGCAAATCCAACATGGTCCATC-3′
and
5′-GATGGACCATGTTGGATTTGCTG-3′,
R725Q
5′-GCTCAGTCTCAGAGCAAATGCTGCGGAG-3′
and
5′-CTCCGCAGCATTTGCTCTGAGACTGAGC-3′,
Q730E
5′-GCTGCGGAGTGAACTACTACTCC-3′
and
5′-GGAGTAGTAGTTCACTCCGCAGC-3′ (Sigma).

T cell activation and MHC I recovery assays. An Erap1 deficient fibroblast cell line was used for all transfection experiments, and B3Z T cell hybridoma were cultured as described previously (1). Erap1 deficient cells were transfected with ERAP1 haplotypes (pcDNA3.1, pcDNA3.1V5/His and/or pcDNA3.1HA) and ES-AIVMK-SHL8 (X5-SHL8) minigene construct (4) using FuGENE 6 (Roche). Presentation of trimmed SHL8 and activation of B3Z T cell hybridoma was assessed as previously described (4). For MHC I recovery, 48 h after transfection Erap1 deficient cells were stained with H-2K^{b} (Y3-FITC) and H-2D^{b} (B22.249-APC) specific antibodies. Cells were analyzed by flow cytometry with a FACS Canto II (BD biosciences) and FlowJo software (TreeStar). The % MHC class I recovery was calculated thus: (mean fluorescence intensity (MFI) of ERAP1 combination—MFI E320A ERAP1)/(MFI WT ERAP1−MFI E320A ERAP1)*100.

Immunoblots. For protein expression, 0.5% NP40 lysates of ERAP1 transfected cells were probed with anti-human ARTS1 (R&D Systems), anti-V5 (Life technologies), anti-HA (Abcam) or anti-glyceraldehyde 3-phosphate dehydrogenase (Abcam) antibodies followed by HRP-conjugated secondary antibody and SuperSignal West Pico or Femto chemiluminescent substrate (Thermo Scientific).

Statistical Analysis, One-way ANOVA with Dunnett's post-test was performed for analysis of differences between multiple groups and control. Fisher's exact test was performed for analysis of differences between the distribution of haplotypes between cases and controls with only haplotypes that had a frequency of greater than 5% of the total number of haplotypes sequenced included (GraphPad prism).

Results and Discussion

### GWAS-Identified Polymorphisms are Functionally Relevant at the Level of Peptide Trimming

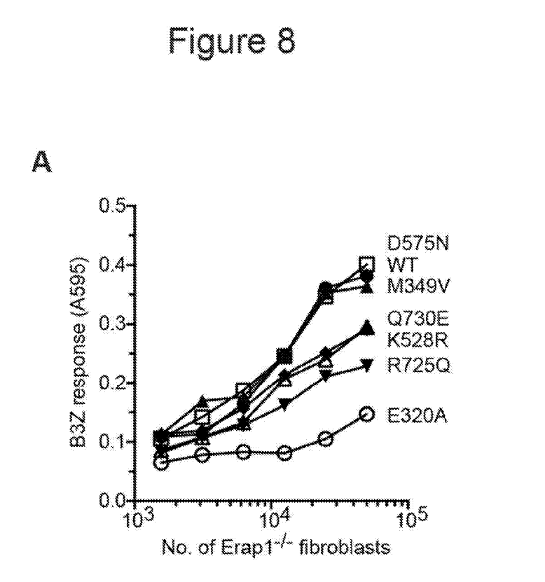

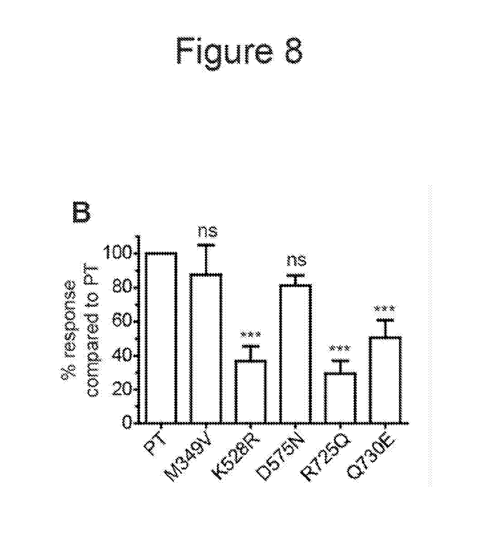

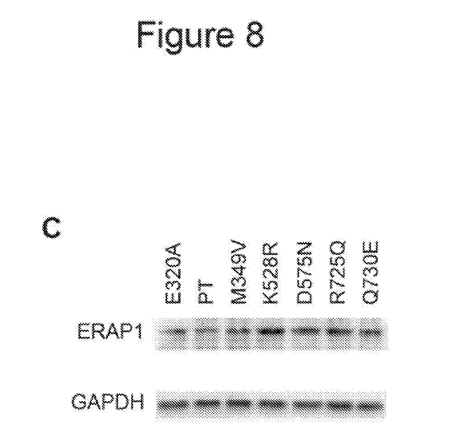

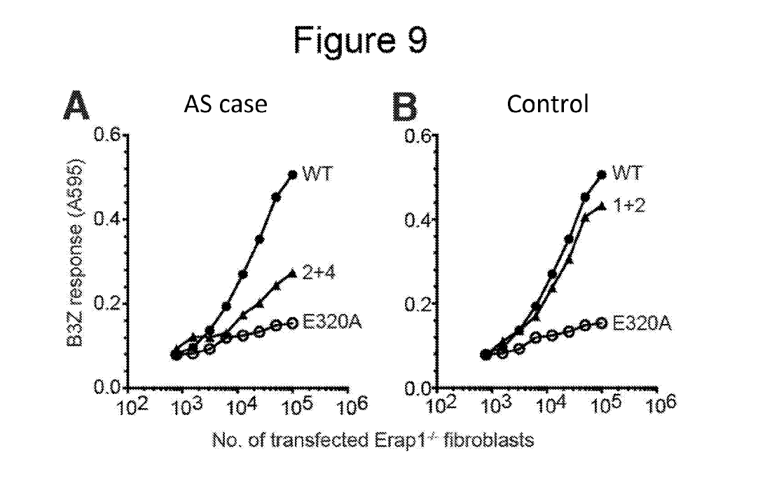

Recent GWAS studies have shown SNPs within ERAP1, M349V (rs2287987), K528R (rs30187), D575N (rs10050860), R725Q (rs17482078) and Q730E (rs27044) to be AS associated, with K528R and Q730E having the strongest linkage. To assess whether disease associated SNPs have an impact on the ability of ERAP1 molecules to trim peptide precursors, we utilized the well characterized SIINFEHL (SHL8) model system, in which an ER targeted five amino acid N-terminally extended precursor AIVMK-SIINFEHL (X5-SHL8) was transfected into Erap1 deficient cells along with ERAP1 (4). Generation of the optimal SHL8 complexed with H-2K^{b} MHC I is monitored by activation of SHL8-specific CD8+ T cells. This assay is specific and sensitive with a detection level of <1 pM SHL8 and has been used previously to illustrate the function of ERAP1 following Erap1 knockout (4). Trimming in Erap1 deficient cells was <90% of that in normal cells but could be restored by transfecting WT (M349, K528, D575, R725 and Q730) ERAP1 (FIG. 8A). ERAP1 containing a single mutation within the active site GAMEN motif (E320A), responsible for N-terminal recognition of peptide substrate, was non-functional and was used as a negative control throughout the study (FIG. 8A). The source of the residual trimming seen in Erap1 deficient cells is unclear but may be from aberrant signal peptidase cleavage or an ERAP1-independent pathway (4). Erap1 deficient cells reconstituted with R725Q, K528R or Q730E single SNP ERAP1 showed a significantly reduced capacity to generate SHL8 from X5-SHL8 compared to WT (reduced by 70%, 63% and 49% respectively; FIGS. 8A and B); M349V and D575N showed no difference to WT. This is similar to previously reported data for K528R, D575N, R725Q and Q730E. The level of expression of ERAP1 molecules was equivalent in all cells (FIG. 8C). This established the validity of the X5-SHL8 minigene model to probe the trimming efficiency of ERAP1.

### ERAP1 Haplotypes Distinguish AS Case Samples from Matched Controls

With the finding that SNPs in ERAP1 affect its trimming ability we undertook to isolate and sequence ERAP1 haplotypes to assess whether particular haplotypes or haplotype combinations are associated with AS. Using molecular cloning we sequenced ERAP1 genes from a cohort of 17 clinically characterized cases and 19 control samples assembled from age and sex-matched cases of non-inflammatory rheumatic illnesses (osteoarthritis, osteoporosis), non-AS inflammatory conditions (rheumatoid arthritis and systemic lupus erythematosus) and healthy volunteers. Samples were tissue typed confirming that all AS cases were HLA-B27 positive (Table V). Upon full-length ERAP1 sequencing, we found that the frequency of haplotypes identified in control samples was very similar to those predicted by HapMap (Table III) with significantly different frequencies to those from AS cases (P<0.05). A minority of ERAP1 sequences deviated from the WT haplotype sequence by one SNP (K528R 14/72 or M349V 1/72; Table III) and 35/72 deviated by two or more SNP combinations that together defined 9 haplotypes (Table III). The WT haplotype was the most prevalent in control samples (50%) and HapMap analysis (44%), whereas this haplotype only represented 9% of those observed in cases. Interestingly, ERAP1 molecules comprising all five AS-linked SNPs, 5SNP, represented 21% of control haplotypes and was the most frequent haplotype in cases accounting for 44% of all molecules identified, but was not represented in the HapMap data. This haplotype has also been identified in the cell line CCRF-CEM and WEWAK-1 confirming that although it was not predicted from HapMap data, it does occur in the population.

We next characterized the haplotype combinations identified in individuals. The majority of samples were heterozygous for ERAP1 and interestingly, no haplotype combination observed in cases was also seen in control samples (Table IV). For example, the 5SNP haplotype, the most prevalent in AS cases, was not found in combination with WT in cases, although this haplotype combination was present in 37% of those identified in controls. Interestingly, the majority of controls (16/19) possessed at least one WT haplotype whereas only a small minority of cases did (3/17). This indicated that AS cases could be distinguished from controls based on their ERAP1 haplotype combination.

### Importance of Combined Haplotypes in Case Cohort: AS Patient ERAP1 Haplotype Combinations Reveal an Overall Reduced Trimming Function

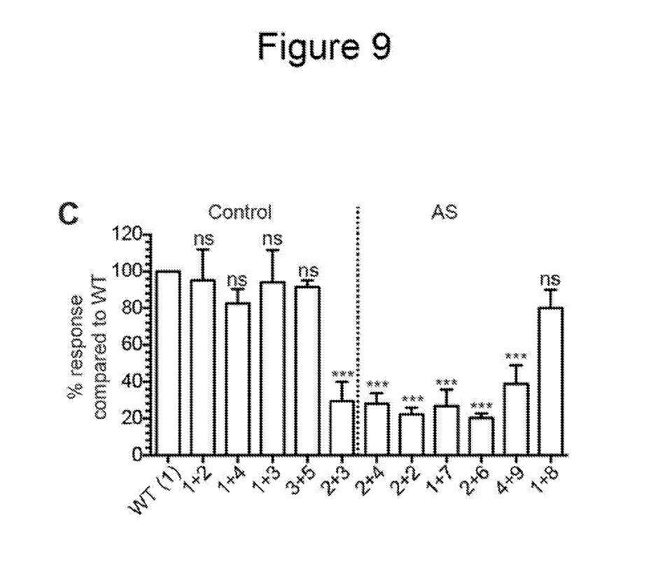

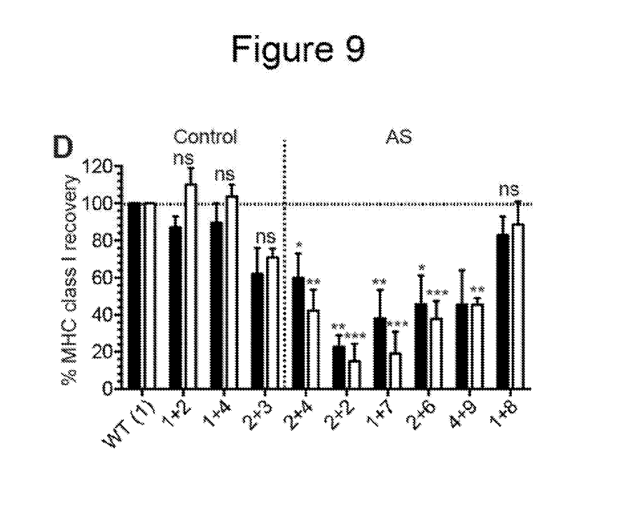

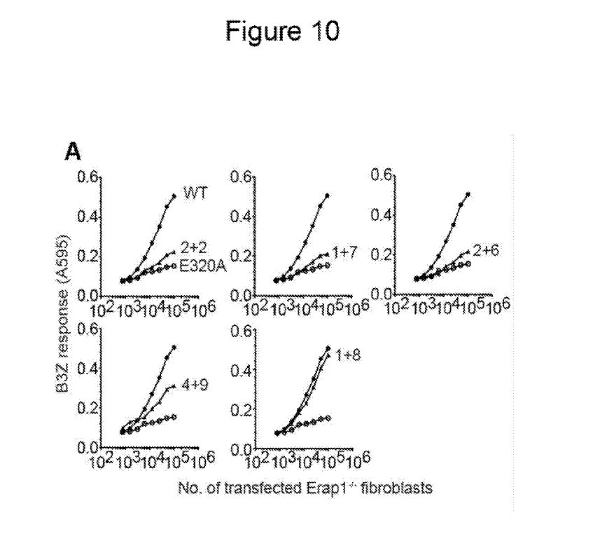

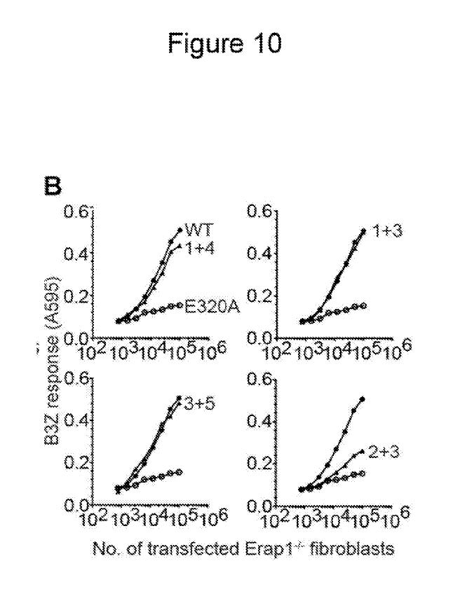

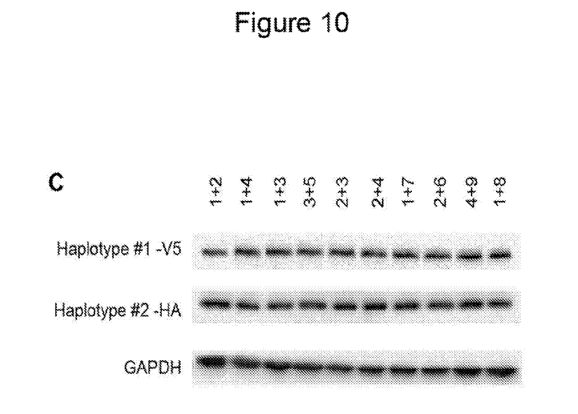

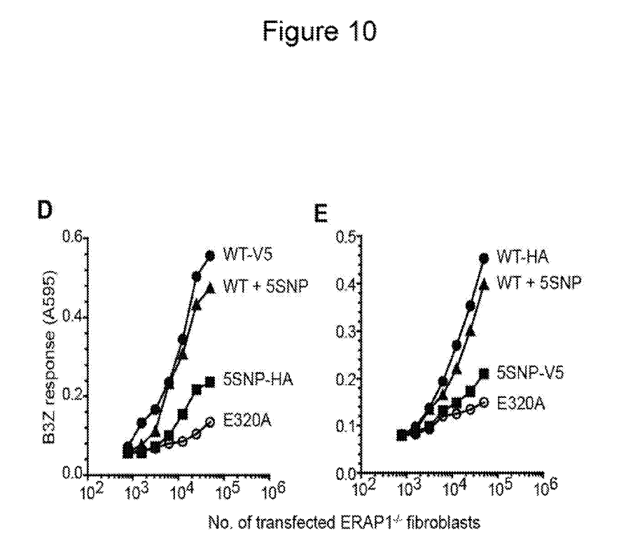

With the ERAP1 haplotype combinations showing clear differences between AS cases and controls we investigated their trimming function. We reconstituted Erap1 deficient cells with pairs of haplotypes corresponding to those combinations identified from individuals and confirmed equivalent expression by western blot (FIG. 10C). The ability of AS case ERAP1 combinations to generate SHL8 from X5-SHL8 was significantly inhibited in most instances (FIGS. 9A and 9C). This was in stark contrast to control haplotype combinations where the predominant trimming function was similar to WT (FIGS. 9B and 9C). Thus, functional discrimination between case and control populations was seen at the level of complete haplotype identity. Interestingly, when 5SNP and K528R are paired with a WT haplotype (as in controls) the trimming function was good (FIGS. 9B and 9C). However, when both 5SNP and K528R haplotypes are combined (as in AS cases) the trimming function was poor (FIGS. 9A and 9C). The observed restoration of a normal trimming function when 5SNP or K528R are co-expressed with the WT haplotype is therefore consistent with a simple loss-of function (FIGS. 9B and 9C and Table IV). In addition, AS case haplotype combinations, in the majority of instances, consist of two haplotypes with poor trimming activities (Table IV and FIG. 9C). However, one combination demonstrated poor trimming capacity even in the presence of a WT haplotype (WT+R725Q/Q730E; FIG. 9C and FIG. 10A). This is consistent with the R725Q/Q730E haplotype having a dominant negative trimming function that may be a consequence of hyperactive trimming activity. To determine whether the observed trimming effects on the index precursor X5-SHL8 could also be seen on the global repertoire of peptides presented, we assessed the ability of ERAP1 combinations to restore cell surface expression of H-2D^{b} and -K^{b} in Erap1 deficient cells to normal levels; Erap1 deficient cells have a 30-40% reduction in MHC. MHC I levels were restored following WT ERAP1 transfection, whereas with E320A ERAP1 transfection MHC I levels were equivalent to vector alone (FIG. 9D). Examination of haplotype combinations in the control group showed the majority were able to restore cell surface MHC I levels (FIG. 9D). Conversely, most disease associated combinations showed significantly reduced MHC I levels (FIG. 9D); the one exception, WT and M349V, showed almost complete restoration. The restoration of MHC I by 5SNP and K528R/Q730E, which was unable to trim X5-SHL8 efficiently, suggests that this combination has a subtle trimming deficiency not applicable to most peptides. The predominant dysfunctional ERAP1 trimming ability observed in AS case haplotype combinations is likely to have a significant impact on the array of peptides generated with hypo-active ERAP1 combinations presenting longer, more unstable, peptides at the cell surface, as shown in the absence of Erap1. Indeed, mass spectrometry analysis of peptides eluted from HLA-B27 in cells with 5SNP ERAP1 has previously revealed the presence of longer peptides compared to WT. Residues 725, 730 and 528 may be important in binding substrates and articulating conformation changes required for catalysis implied by structural studies.

ERAP1 trimming phenotype may impact on the biochemistry and antigen presenting function of HLA-B27. The formation of HLA-B27 homodimers (B27₂) in the ER and at the cell surface has been implicated in the pathogenesis of AS through either the induction of the unfolded protein response (UPR) in the ER, or activation of innate cells at the cell surface. B27₂ formation in the ER and at the cell surface is promoted in conditions where the availability of optimal peptides or peptide editing is suboptimal (TAP^{-/-}, TPN^{-/-} and ERAP1 knockdown), and our data show that naturally occurring ERAP1 variants may lead to the restricted supply of optimal peptides. Differences in ERAP1 trimming phenotypes may alter the abundance of some peptides contributing to disease pathogenesis similar to that suggested by the arthritogenic peptide hypothesis.

This study shows how ERAP1 function could impact on disease pathogenesis and how elucidation of distinct haplotype combinations in AS cases provides biomarkers for disease stratification.

Tables VII to XII provide data for other conditions, showing that ERAP1 haplotype analysis may also be used for diagnosis of those conditions.

### REFERENCES

1. Hammer, G. E., F. Gonzalez, M. Champsaur, D. Cado, and N. Shastri. 2006. The aminopeptidase ERAAP shapes the peptide repertoire displayed by major histocompatibility complex class I molecules. Nature immunology 7: 103-112.
2. Serwold, T., S. Gaw, and N. Shastri. 2001. ER aminopeptidases generate a unique pool of peptides for MHC class I molecules. Nature immunology 2: 644-651.
3. Kanaseki, T., and N. Shastri. 2008. Endoplasmic reticulum aminopeptidase associated with antigen processing regulates quality of processed peptides presented by MHC class I molecules. Journal of immunology 181: 6275-6282.
4. Kanaseki, T., N. Blanchard, G. E. Hammer, F. Gonzalez, and N. Shastri. 2006. ERAAP synergizes with MHC class I molecules to make the final cut in the antigenic peptide precursors in the endoplasmic reticulum. Immunity 25: 795-806.
5. Malarkannan, S., S. Goth, D. R. Buchholz, and N. Shastri. 1995. The role of MHC class I molecules in the generation of endogenous peptide/WIC complexes. Journal of immunology 154: 585-598.
6. Truscott, S. M., L. Lybarger, J. M. Martinko, V. E. Mitaksov, D. M. Kranz, J. M. Connolly, D. H. Fremont, and T. H. Hansen. 2007. Disulfide bond engineering to trap peptides in the MHC class I binding groove. Journal of immunology 178: 6280-6289.

### EXAMPLE 3

### Further Work

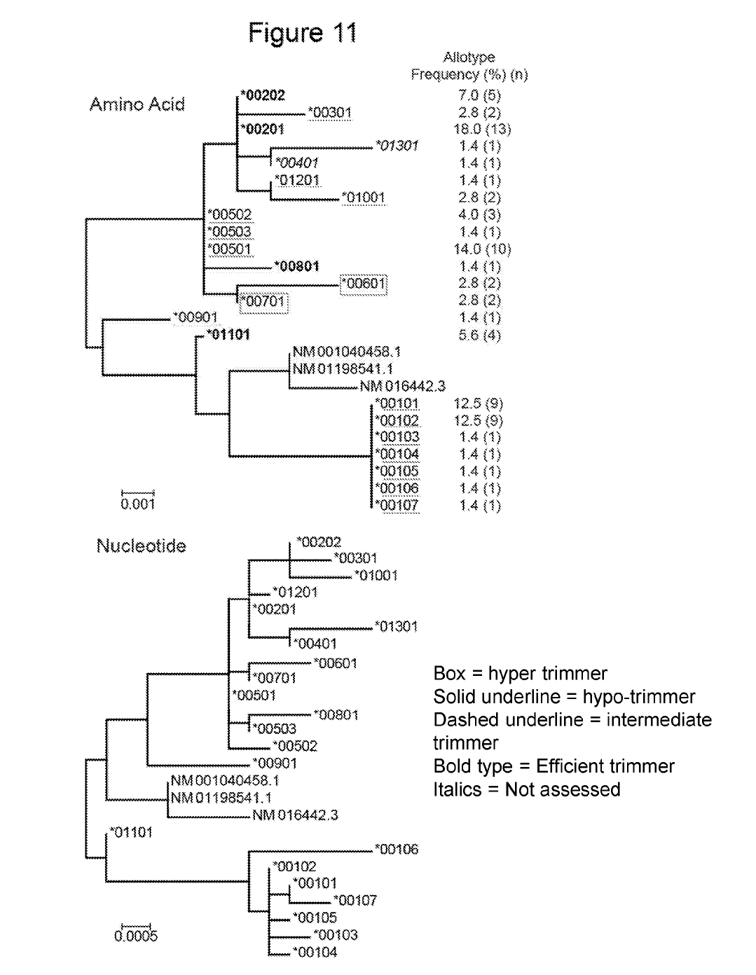

This Example concerns further work and overlaps in part with the previous Examples: we have previously shown that ERAP1 exists as distinct allotypes within individuals with the majority of allotypes consisting of at least two AS-associated polymorphisms. Given the association of ERAP1 SNPs with AS, we therefore wanted to investigate whether particular ERAP1 allotypes were associated with AS. To this end we isolated the full length coding sequence of ERAP1 from AS cases and controls. Using molecular cloning we sequenced ERAP1 genes from a cohort of 17 clinically characterized cases and 19 control samples assembled from age and sex-matched cases of non-inflammatory rheumatic illnesses (osteoarthritis, osteoporosis), non-AS inflammatory conditions (rheumatoid arthritis and systemic lupus erythematosus) and healthy volunteers. Samples were tissue typed confirming that all AS cases were HLA-B27 positive. Analysis of the full-length ERAP1 coding sequence revealed 13 distinct allotypes based on amino acid sequence. The allotypes were found to contain multiple polymorphisms, which included the five SNPs associated with AS (Table XIV). Further investigation revealed a number of conservative nucleotide variations, which, although not changing protein sequence, further delineated ERAP1 molecules (Table XV). As ERAP1 is highly polymorphic (13 different allotypes (22 difference sequences) identified from 36 individuals) we undertook to standardize the ERAP1 allotype sequence nomenclature to allow better and clearer documentation and discrimination of identified ERAP1 allotypes. To this end we established the nomenclature ERAP1*000:00:00, where the first group of three digits identifies ERAP1 molecules with coding amino acid differences defining the distinct allotypes. The second group of digits denotes variation within allotypes that represent conservative nucleotide changes. The final group of digits discriminate molecules within allotypes that have variation in intronic and/or untranslated regions (5′ and 3′ UTR; which were not examined in this study). We applied this standardizing nomenclature to the ERAP1 allotypes we identified from our cohort and listed the amino acid positions where variation between allotypes was most frequent and their identity (Table XIV). The greatest extent of amino acid variation was between allotypes ERAP1*001 and *002 which have 13 differences throughout the coding sequence including five previously described non-synonymous polymorphisms at amino acid positions 349, 528, 575, 725 and 730. Most of the other sequences had varying combinations of these differences making up the allotypes (Table XIV). We identified three allotypes with additional diversity in conservative nucleotides, the greatest being for allotype ERAP1*001 where 7 sub-types were identified, perhaps reflecting its high frequency in the population (Tables XIV and XV). Most allotypes contained at least one of the previously described SNPs. In addition, we found non-synonymous SNPs that have not been described previously at amino acid positions 82, 102, 115, 581, 737 and 752; and others at previously described positions but encoding different amino acids (F199C, L727P and M874T). These novel polymorphisms made up the majority of the differences between ERAP1*001 and *002 allotypes. To further assess the relationship between identified ERAP1 allotypes we performed phylogenetic analysis of the identified nucleotide and amino acid sequences (FIG. 11). The resultant unrooted phylogenetic trees reveal two major branches with the six loci (82, 102, 115, 199, 581, 737) important in this discrimination. We have previously described functional variation among ERAP1 encoded by nine different allotypes which broadly fell into three functional groups: “normal”, “hypo” and “hyper” trimmers. When this trimming function is superimposed on the phylogenetic tree of amino acid sequences (FIG. 11), we found evidence of clustering of functionally similar allotypes. Intriguingly, the hyper-active ERAP1*006 and *007 are closely related to the hypo-active *005 and normal *008 allotypes only varying at one or two loci. The hyper-active allotypes contain a Q725 polymorphism whereas the normal allotypes do not, indicating that Q725 is important in the acquisition of a hyper-active trimming phenotype.

### ERAP1 Allotypes Distinguish AS Case Samples from Matched Controls

Using the new nomenclature we determined the ERAP1 allotypes identified from AS cases (n=34) and controls (n=38; Table XIV). Some allotypes were found to be more prevalent in controls (ERAP1*002 and *011) whereas others were more prevalent in cases (ERAP1*001 and *005). Interestingly, the most frequent allotypes in both control and case groups were ERAP1*002 and *001, which are the most divergent with respect to amino acid differences (13 changes). Moreover, previous assessment of the trimming function of these ERAP1 molecules showed that allotype *002 trimmed peptide precursors efficiently whereas allotype *001 was hypo-active. Analysis of the second most frequent case allotype, ERAP1 *005, showed that the trimming function was reduced for peptide precursors; K528R and below. Thus, although there appeared to be some association between allotype and disease, this association was not evident at the level of ERAP1 function.

Since both chromosomal copies of ERAP1 are co-dominantly expressed, we next determined the combinations of allotype in our AS cohort and control group. Interestingly, the majority of samples were heterozygous for ERAP1 (32/36) and strikingly, no allotype pair observed in cases was also seen in control samples (Table XVI). For example, the *001 allotype, the most prevalent in AS cases, was not found in combination with *002 in cases, although this allotype pair was present in about a third (37%) of those identified in controls. Furthermore, the *002 allotype was observed in most of the controls (15/19), but in only one case (1/17). This indicated that AS cases could be distinguished from controls based on their ERAP1 allotype combination.

### Importance of Combined Allotypes in Case Cohort: AS Patient ERAP1 Allotype Pairs Reveal an Overall Reduced Trimming Function

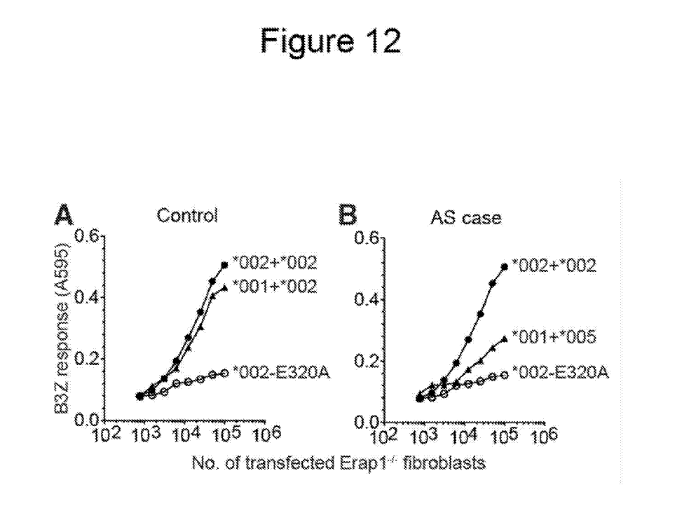

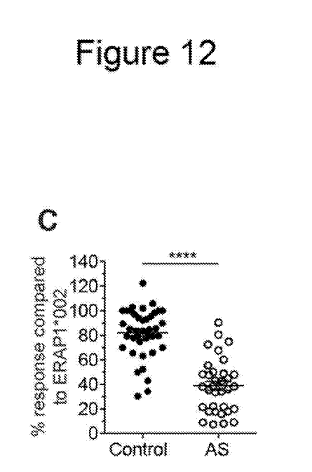

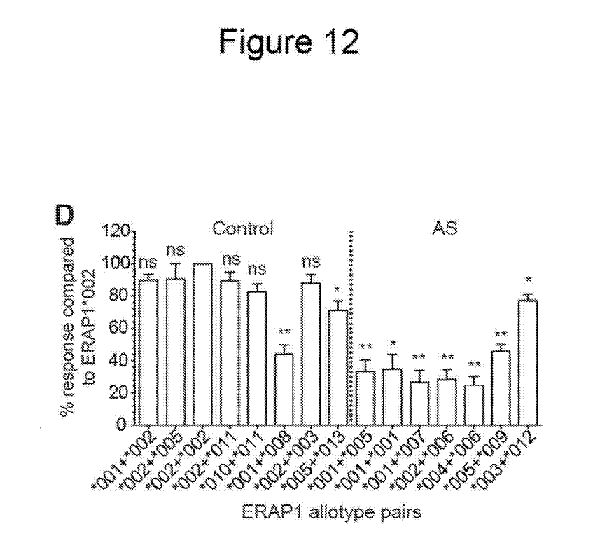

With the ERAP1 allotype pairs showing clear differences between AS cases and controls we investigated whether the combined trimming functions of co-dominantly expressed ERAP1 molecules were also different. We chose to measure the trimming function of ERAP1 in situ in the antigen processing pathway of living cells using a well characterized assay, which we have previously used, to measure function of ERAP allotypes and allotype pairs. The assay reports the generation of an epitope, SIINFEHL (SHL8), from an ER targeted five amino acid N-terminally extended precursor (AIVMK-SIINFEHL or X5-SHL8) encoded by a minigene which was transfected into Erap1 deficient cells along with ERAP1. Generation of the optimal SHL8 complexed with H-2K^{b} MHC I is monitored by activation of SHL8-specific CD8+ T cells and is sensitive to <1 pM. Trimming in Erap1 deficient cells was <90% of that in normal cells but could be restored by transfecting ERAP1*002 (FIG. 12A). ERAP1*002 containing a single mutation within the active site GAMEN motif (E320A), responsible for N-terminal recognition of peptide substrate, was non-functional and was used as a negative control throughout the study (FIG. 12A). The source of the residual trimming seen in Erap1 deficient cells is likely to be from aberrant signal peptidase cleavage or an ERAP1-independent pathway, but does not interfere with the assay other than to raise the background level. We reconstituted Erap1 deficient cells with pairs of allotypes corresponding to those combinations identified from individuals and confirmed equivalent expression by western blot. The ability of AS case ERAP1 combinations to generate SHL8 from X5-SHL8 was significantly reduced in most instances (FIG. 12B-D). This was in stark contrast to control allotype combinations where the predominant trimming function was similar to homozygous ERAP1*002 allotypes (FIGS. 12A, C and D). The difference in ability to trim peptide precursor is most evident when comparing all responses observed between control and AS case allotype pairs, where AS group trimming function was ˜50% of that of the controls (FIG. 12C). Thus, discrimination between case and control populations was seen at the level of function only when the combined function of both co-dominantly expressed ERAP1 allotypes were analyzed. Interestingly, when ERAP1*001 or *005 are paired with a *002 allotype (as in controls) the trimming function was good (FIG. 12D). However, when both ERAP1*001 and *005 allotypes are combined (as in AS cases) the trimming function was poor (FIGS. 12A and D). The observed restoration of a normal trimming function when ERAP1*001 or *005 are co-expressed with ERAP1*002 is therefore consistent with a simple loss-of function (FIG. 12D and Table XVI). The majority of allotype pairs from AS cases consisted of two allotypes with poor trimming activities (Table XVI). Where hypo-active allotypes appeared in the control group, they were always paired with a normal functioning allotype; for example the relatively frequent pairing of ERAP1*001 with ERAP1*002. Conversely, when normal functioning allotypes appeared in the AS case cohort, they were paired with allotypes that in combination demonstrated poor trimming capacity; for example ERAP1*002 paired with *006 (FIG. 12D). This is consistent with ERAP1*006 allotype being hyper-active and thus exerting a dominant negative trimming function.

### Affect of ERAP1 Allotype Combinations on Peptide Repertoire and MHC I Expression

We have previously shown that while X5-SHL8 is an informative index substrate for broadly classifying ERAP1 function, fine substrate specificity is also observed among ERAP 1 variants. To determine whether the observed trimming effects of X5-SHL8 was a fair representation of more global trimming function, we assessed the ability of ERAP1 pairs to restore cell surface expression of H-2D^{b} and -K^{b} in Erap1 deficient cells to normal levels; Erap1 deficient cells have a 20-30% reduction in MHC which was restored to normal levels following ERAP1*002 transfection (FIG. 9D). We measured the ability of allotype pairs to restore MHC I expression and plotted the results as a direct comparison with the effect of the ERAP1*002 transfectants. All allotype pairs found in the control group were able to restore cell surface MHC I levels (FIG. 9D). Conversely, most disease associated pairs were unable to restore MHC I levels (FIG. 9D; >50% reduction). We noted one exception which was the combination of ERAP1*003 and *012 found in one of our AS cases and which induced almost complete restoration. The reason for this is still unclear, but we are investigating the fine specificity of this combination incase, for example, it gives rise to a rare H2-binding peptide. In support of this idea we found that this combination did not restore HLA-B27 expression in the same way (see below).

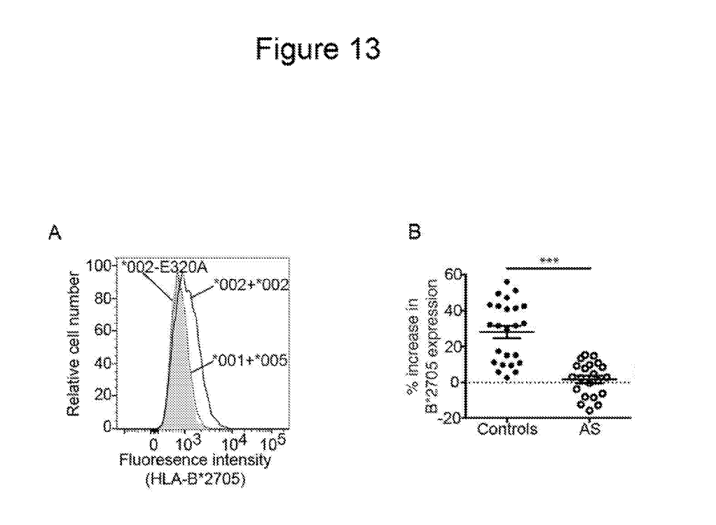

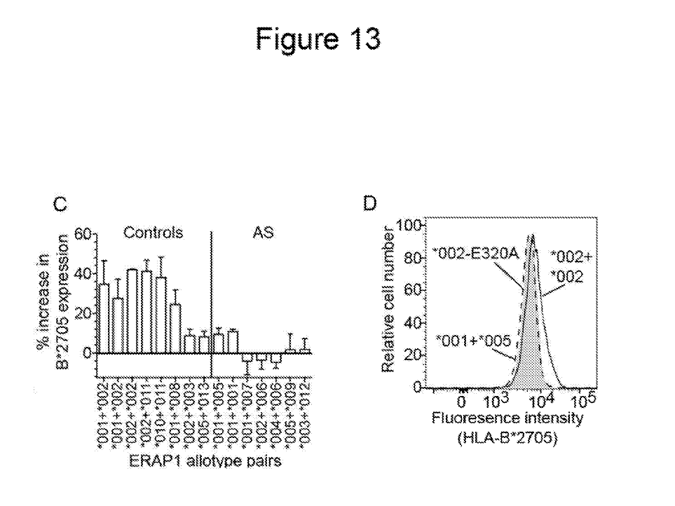

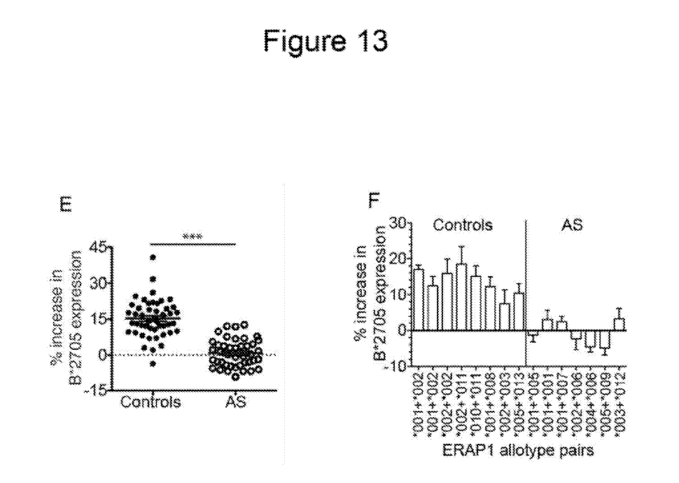

HLA-B*27:05 is the most prevalent HLA-B27 subtype associated with AS and was expressed by all AS patients in our cohort. We therefore investigated the effect of ERAP1 pairs on HLA-B*27:05 (hereafter referred to as HLA-B27) cell surface expression. Erap1 deficient cells were transfected with HLA-B27, human β₂M and the ERAP1 combinations and the expression of HLA-B27 examined. Control ERAP1 pairs show a significant increase in HLA-B27 levels compared to AS cases (28% versus 2%; FIGS. 13A and B). Examination of the effect of individual ERAP1 pairs on HLA-B27 levels revealed that the majority of control ERAP1 pairs showed a 20-40% increase in HLA-B27 cell surface expression (FIGS. 13B and C). Two ERAP1 combinations (*002+*003 and *005+*013) showed a modest increase of <10% compared to the nonfunctional *002-E320A ERAP1 (FIG. 13C). By contrast, all of the ERAP1 pairs from AS individuals showed very little difference in B27 expression compared to nonfunctional negative control, with some even decreasing in HLA-B27 levels further (FIGS. 13B and C). These data suggest that, similar to that observed for H-2D^{b} and -K^{b}, the ERAP1 pairs found in AS cases generate fewer peptides capable of stabilizing B*2705 compared to combinations found in controls.

To further investigate the effect of ERAP1 combinations on HLA-B27 cell surface levels we utilized an ERAP1 KO 293T human cell line. This cell line was created using the CRISPR/Cas9 system to target ERAP1 and introduce a double stranded nick, which, following repair, introduced frame shift mutations resulting in premature stops in both copies of ERAP1. These ERAP1 KO 293T cells do not produce any detectable ERAP1 protein and fail to trim X5-SHL8 precursor when transfected. 293T ERAP1 KO cells expressing HLA-B27 were transfected with ERAP1 pairs and their effect on HLA-B27 levels assessed. The control ERAP1 pairs showed a significant increase in HLA-B27 compared to AS case ERAP1 pairs (15% versus 1%; FIGS. 13D and E). Examination of individual ERAP1 combinations revealed that all those identified in controls increased HLA-B27 levels by 10-20% (FIG. 13F). By contrast, only 3 of the 7 AS case ERAP1 pairs identified increased HLA-B27 cell surface expression, albeit at a low level (<5%), with HLA-B27 levels reduced in the other combinations (up to −5%; FIG. 13F). This further confirmed that AS case ERAP1 pairs generate fewer HLA-B27 stabilizing peptide ligands. It is therefore likely that the repertoire of peptides presented at the cell surface is significantly different between cases and controls.

Discussion

In this study we have shown that ERAP1 is highly polymorphic with 13 distinct allotypes assembled from at least 15 non-synonymous nucleotide variants identified from 36 genomes. Our analysis of the complete coding sequence revealed a further nine polymorphic variants, three of which have been previously observed coding for different amino acids (199, 727 and 874). Interestingly, phylogenetic analysis revealed six of the novel variants (82, 102, 115, 199, 581, 737) formed the basis for the main branch point of ERAP1 (FIG. 11). In almost all allotypes identified (71/72), the six variants were co-inherited forming a backbone, suggestive of an early evolutionary branching based on these variants. Many studies have identified and confirmed the linkage between ERAP1 SNPs and disease risk in different populations; the strongest linkage found at residues 528 and 730, which are found in allotypes in case and control groups. K528/D575/E730 represented by ERAP1*006 was only present in AS cases, albeit at low frequency (Tables XIV and XVI). In contrast to previous reported work, we revealed perfect stratification of AS patients and controls when both allotypes present in a single individual's genome were examined: leading to the identification of combinations associated with disease. Moreover, this stratification was rationalized at the functional level since AS associated allotype pairs encoded ERAP1 with poor trimming function.

We have shown previously that hyperfunctional ERAP1 allotypes lead to inefficient generation of optimal peptide epitopes using N-terminally extended substrates and lead to changes in the peptide repertoire by mass spectrometry analysis, which is likely to result in the reduction of cell surface HLA-B27 expression. AS case allotype pairs 11, 12 and 13 all contained allotypes with a hyperactive trimming phenotype (*006 and *007) which failed to restore MHC I expression (H-2K^{b}, -D^{b} or HLA-B*2705) in ERAP1 knockout cells even when co-expressed with a normal function ERAP1 consistent with the dominant negative effect we have previously shown.

The mechanism by which differences in ERAP1 primary structure contribute to the differences in function we observe is not clear. Four of the six (82, 102, 115 and 199) together with residue 127 are located in domain I away from the active site. Interestingly, the end of the S1 specificity pocket in domain 11 borders residues 181 and 183 in domain I and therefore the observed polymorphisms may affect the formation of the catalytic site (19, 28). In addition, residue 127 may affect conformational transition from open to closed states as previously proposed. Similarly, the AS associated residue 528 is likely to affect the ability to adopt a correct catalytic conformation as it lies in a region of the molecule that has been proposed to articulate the conformational change. Residue 581 is situated in a β-strand in domain III and similarly to residue 575 (closely located as part of a loop), may affect flexibility of domain III (26). Residue 349 is close to the active site and therefore may affect trimming. By contrast, residue 737 forms part of an α-helix also containing the AS associated residues 725 and 730 (and the 727 novel variant) in domain IV. These residues are located within the substrate binding cavity, which may interact with the C-terminus of peptide substrates as part of the “regulatory” domain and therefore may alter the binding and/or trimming specificity of ERAP1.

Although it is not known why ERAP1 is so polymorphic, the identification of an ERAP1 trimming resistant HIV gag epitope and targeting of ERAP1 by human cytomegalovirus indicates selective pressure from infectious agents/pathogens similar to, but to a lesser extent than, that observed for HLA (MHC). One consequence of increased genetic diversity in ERAP1 could be that the evolution of allotypes that confer better protection to a particular pathogen may, when expressed in individuals of particular HLA types such as B*2705 and B*5701, predispose these individuals to autoimmune disease.

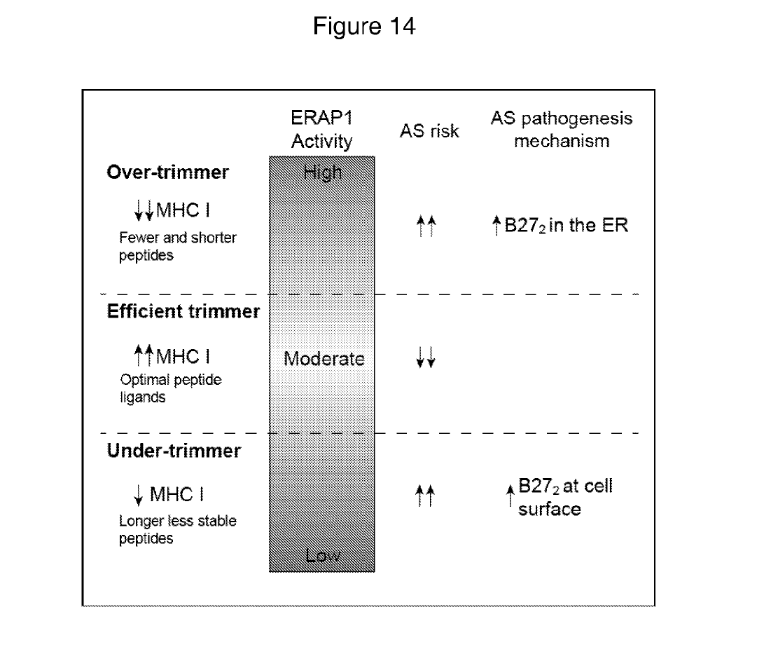

Altered ERAP1 activity leading to a change in peptide repertoire impacts on the biochemistry and antigen presenting function of HLA-B27. Based on our findings we propose a model which links the relative activity of ERAP1 variants to disease via its likely effect on biochemical features of HLA-B27 that have been previously implicated in AS. HLA-B27 has a propensity to form heavy chain homodimers (B27₂) either in the ER as a result of limited peptide supply or impaired peptide selection; or at the cell surface as a result of peptide dissociation; (B27₂) formed in the ER do not traffic to the cell surface. (B27₂) have thus been implicated in the pathogenesis of AS through two different mechanisms: either the induction of the unfolded protein response (UPR) in the ER, or activation of innate and/or Th17 cells through KIR3DL2 engagement at the cell surface. Our data unify these mechanisms based on an understanding of ERAP1 function since ERAP1 variants with high trimming activity may lead to the restricted supply of optimal peptides (FIGS. 12 and 13) predisposing to the formation of ER B27₂ and UPR; whereas, B27₂ formation at the cell surface may be promoted by hypofunctional ERAP1 which generate longer peptides that despite binding to HLA-B27 with sufficient affinity to pass intracellular quality control, nevertheless dissociate rapidly at the cell surface leading to increased B27₂ there. These mechanisms are not necessarily mutually exclusive, nor do they preclude other possible mechanisms such as the ability of different ERAP1 variants to generate specific arthritogenic peptides (FIG. 14).

Finally, the ERAP1 homologue ERAP2 have also been linked with AS and a change in trimming function. The mouse genome does not contain an orthologue of ERAP2. We found little difference in the re-expression phenotype of HLA-B27 in murine Erap1−/− cells versus ERAP1 KO 293T cells suggesting that any effect ERAP2 has on peptide generation is small. This supports the idea that ERAP1 is the main component of peptide trimming in cells. Nevertheless, it remains to be determined whether ERAP2 molecules in 293T cells are a low activity variant, and our ERAP1 KO 293T cells provide a tool for investigating the identity and function of ERAP2 expressed in these cells (and other variants) in the absence of ERAP1.

In conclusion, this study how ERAP1 function impacts on disease pathogenesis and how the distinct allotype combinations we have described in AS cases, may serve as biomarkers for disease stratification and a novel target for treatment.

Tables XVII, XXIII and XXIV show how the new nomenclature relates to the old nomenclature. Tables XVIII to XXII show data for other conditions.

TABLE I
Identity of ERAP1 haplotypes in the samples studied.

| | | SNP | | | | |
|---|---|---|---|---|---|---|
| | | rs2287987 | rs30187 | rs10050860 | rs17482078 | rs27044 |
| | | (M349V) | (K528R) | (D575N) | (R725Q) | (Q730E) |
| Haplotype | WT | t/M | t/K | c/D | c/R | g/Q |
| | 5SNP | c/V | c/R | t/N | t/Q | c/E |
| | K528R/Q730E | t/M | c/R | c/D | c/R | c/E |
| | K528R | t/M | c/R | c/D | c/R | g/Q |
| | M349V/D575N/ | c/V | t/K | t/N | t/Q | g/Q |
| | R725Q | | | | | |
| | K528R/R725Q | t/M | c/R | c/D | t/Q | g/Q |
| | R725Q/Q730E | t/M | t/K | c/D | t/Q | c/E |
| | M349V | c/V | t/K | c/D | c/R | g/Q |
| | M349V/K528R | c/V | c/R | c/D | c/R | g/Q |

| | | | | | |
|---|---|---|---|---|---|
| AS association | 1.4 × 10⁻⁴ | 4.9 × 10⁻⁶ | 1.2 × 10⁻⁴ | 4.0 × 10⁻⁵ | 1.6 × 10⁻⁶ |
| (P-value) | | | | | |
| Frequency % | 77/23 | 32/68 | 75/25 | 76/24 | 26/74 |
| (WT/variant) | | | | | |

Lower case letter denotes anti-sense strand base pair and upper case letter denotes the amino acid at this position.

TABLE II
Relative amino acid trimming efficiency compared to WT ERAP1

| | SHL8 generation compared to WT from X-SHL8 | | | Total SHL8 |
|---|---|---|---|---|
| Allele | 75-100% | 50-75% | 0-50% | generation⁺* |
| K528R/R725Q | F, M, A, P | N | E, H, K, T, I, R, V, | 345.4 |
| | | | L, C, D, W, Q, S | |
| 5SNP | Q, P | M, V | L, E, A, W, H, T, I, | 366.9 |
| | | | N, R, F, C, D, K, S | |
| R725Q/Q730E | P | M, A, W, I, R | V, L, D, E, H, K, T, | 378.7 |
| | | | S, C, Q, N, F | |
| M349V/D575N/ | P | M, A, Q, S, R | V, L, E, W, H, K, | 383.9 |
| R725Q | | | T, I, C, D, N, F | |
| M349V | R, M, Q, I | V, E, A, K, T | L, D, W, N, S, C, | 436.9 |
| | | | H, F, P | |
| M349V/K528R | Q, I, R, F | M, L, D, E, A, H, T, N, S | V, W, K, C, P | 533.6 |
| K528R/Q730E | H, R, F, P, M, W, | V, L, E, K, Q, I | C, D, A | 691.6 |
| | T, N, S | | | |
| K528R | L, A, W, K, S, R, | V, D, H | C | 862.6 |
| | F, P M, E, Q, T, I, N | | | |

⁺Total SHL8 generation is the sum of SHL8 generated from all N-terminal amino acids.
*WT = 950.7

TABLE III
Identity and frequency of ERAP1 haplotypes in the populations studied.

| | | | | | | Frequency | |
|---|---|---|---|---|---|---|---|
| | SNP | | | | | Control | Case |
| | rs2287987 | rs30187 | rs10050860 | rs17482078 | rs27044 | (n = 38) n | (n = 34) n |
| Haplotype | (M349V) | (K528R) | (D575N | (R725Q) | (Q730E) | (%) | (%) |
| 1 (WT) | T | T | C | C | G | 19 (50) | 3 (9) |
| 2 (5SNP) | C | C | T | T | C | 8 (21) | 15 (44) |
| 3 (K528R/Q730E) | T | C | C | C | C | 5 (13) | 0 |
| 4 (K528R) | T | C | C | C | G | 4 (11) | 10 (29) |
| 5 (M349V/D575N/ | C | T | T | T | G | 2 (5) | 0 |
| R725Q) | | | | | | | |
| 6 (K528R/R725Q) | T | C | C | T | G | 0 | 2 (6) |
| 7 (R725Q/Q730E) | T | T | C | T | C | 0 | 2 (6) |
| 8 (M349V) | C | T | C | C | G | 0 | 1 (3) |
| 9 (M349V/K528R) | C | C | C | C | G | 0 | 1 (3) |

Bold type indicates altered SNP compared to WT.

TABLE IV
Identity and frequency of ERAP1 haplotype combinations in the
populations studied.

Frequency

| Haplotype | Controls (n = 19) | Case (n = 17) |
|---|---|---|
| combination* | n (%) | n (%) |
| 1 + 2 | 7 (37) | 0 |
| 1 + 4 | 4 (21) | 0 |
| 1 + 1 | 3 (16) | 0 |
| 1 + 3 | 2 (11) | 0 |
| 3 + 5 | 2 (11) | 0 |
| 2 + 3 | 1 (5) | 0 |
| 2 + 4 | 0 | 9 (53) |
| 2 + 2 | 0 | 2 (12) |
| 1 + 7 | 0 | 2 (12) |
| 2 + 6 | 0 | 2 (12) |
| 4 + 9 | 0 | 1 (6) |
| 1 + 8 | 0 | 1 (6) |

*1 = WT, 2 = 5SNP, 3 = K528R/Q730E, 4 = K528R, 5 = M349V/D575N/R725Q, 6 = K528R/R725Q, 7 = R725Q/Q730E, 8 = M349V, 9 = M349V/K528R.

TABLE V
Characteristics of patients recruited for the study.
All patients had confirmed Axial SpA diagnosis with
15/17 confirmed AS using New York criteria.

| | | | Diagnosis | Diagnosis | |
|---|---|---|---|---|---|
| Patient | Gender | Age | (ASAS) | (New York) | HLAB27 |
| 1 | M | 46 | Yes | Yes | + |
| 2 | M | 62 | Yes | Yes | + |
| 3 | M | 45 | Yes | Yes | + |
| 4 | M | 64 | Yes | Yes | + |
| 5 | M | 37 | Yes | Yes | + |
| 6 | M | 48 | Yes | No | + |
| 7 | F | 38 | Yes | Yes | + |
| 8 | M | 54 | Yes | Yes | + |
| 9 | M | 54 | Yes | Yes | + |
| 10 | F | 61 | Yes | Yes | + |
| 11 | M | 64 | Yes | Yes | + |
| 12 | M | 28 | Yes | Yes | + |
| 13 | M | 53 | Yes | Yes | + |
| 14 | F | 32 | Yes | Yes | + |
| 15 | M | 26 | Yes | Yes | + |
| 16 | F | 42 | Yes | No | + |
| 17 | F | 65 | Yes | Yes | + |

Controls were all patients attending the rheumatology department with a non-inflammatory illness or healthy volunteers.

TABLE VI
Additional Polymorphisms

| | Found with 5SNP | Found with |
|---|---|---|
| Polymorphism | haplotype | M349V/K528R |
| I82V | YES | YES |
| L102I | YES | YES |
| P115L | YES | YES |
| R127P | YES | YES |
| S199F | YES | YES |
| S581L | YES | |
| L727A | | |
| A737V | YES | |

L727A is only present in a small subset of WT haplotypes

TABLE VII
Psoriasis data

| | Frequency |
|---|---|
| Haplotype | (n = 14) |
| WT | 3 |
| M349V | 7 |
| K528R | 1 |
| K528R/Q730E | 3 |

TABLE VIII
Psoriasis data

| Haplotype | Frequency |
|---|---|
| combination | (n = 5) |
| WT + M349V | 2 |
| WT + K528R | 1 |
| WT + K528R/Q730E | 1 |
| M349V + | 1 |
| K528R/Q730E | |

TABLE IX
Type-1-diabetes data

Frequency

| | | T1D Cases |
|---|---|---|
| Haplotype | Controls (n = 9) | (n = 8) |
| WT | 3 | 4 |
| 5SNP | 1 | 1 |
| R127P | 1 | 0 |
| K528R/Q730E | 4 | 2 |
| K528R | 0 | 1 |

TABLE X
Type-1-diabetes data

| | |
|---|---|
| Haplotype | Frequency |

| combination | Controls (n = 3) | T1D Cases (n = 2) |
|---|---|---|
| WT + | 1 | 1 |
| K528R/Q730E | | |
| K528R/Q730E + | 1 | 0 |
| K528R/Q730E | | |
| WT + 5SNP | 1 | 1 |

TABLE XI
Head and Neck Squamous Cell Carcinoma (HNSCC) data

| Haplotype | Frequency (n = 36) |
|---|---|
| WT | 8 |
| 5SNP | 6 |
| K528R/Q730E | 3 |
| K528R/D575N/Q730E | 4 |
| M349V/R725Q/Q730E | 2 |
| D575N | 2 |
| R725Q | 1 |
| K528R/D575N | 2 |
| M349V/K528R/D575N/ | 2 |
| Q730E | |
| R725Q/Q730E | 2 |
| K528R/R725Q | 1 |
| Q730E | 2 |
| K528R/R725Q/Q730E | 1 |

TABLE XII
Head and Neck Squamous Cell Carcinoma (HNSCC) data

| | Frequency |
|---|---|
| Haplotype combination | (n = 14) |
| WT + 5SNP | 3 |
| WT + K528R/Q730E | 1 |
| WT + R725Q | 1 |
| M349V/R725Q/Q730E + Q730E | 2 |
| WT + D575N | 2 |
| M349V/K528R/D575N/Q730E + K528R/D575N | 2 |
| WT + K528R/D575N/Q730E | 1 |
| K528R/D575N/Q730E + K528R/R725Q | 1 |
| 5SNP + K528R/R725Q/Q730E | 1 |

TABLE XIII
ERAP1 Sequences

ERAP1 WT-nucleotide sequence:
SEQ ID NO: 1
ATGGTGTTTCTGCCCCTCAAATGGTCCCTTGCAACCATGTCATTTCTAC
TTTCCTCACTGTTGGCTCTCTTAACTGTGTCCACTCCTTCATGGTGTCA
GAGCACTGAAGCATCTCCAAAACGTAGTGATGGGACACCATTTCCTTGG
AATAAAATACGACTTCCTGAGTACGTCATCCCAGTTCATTATGATCTCT
TGATCCATGCAAACCTTACCACGCTGACCTTCTGGGGAACCACGAAAAT
AGAAATCACAGCCAGTCAGCCCACCAGCACCATCATCCTGCATAGTCAC
CACCTGCAGCTATCTAGGGCCACCCTCAGGAAGGGAGCTGGAGAGAGGC
CATCGGAAGAACCCCTGCAGGTCCTGGAACACCCCCGTCAGGAGCAAAT
TGCACTGCTGGCTCCCGAGCCCCTCCTTGTCGGGCTCCCGTACACAGTT
GTCATTCACTATGCTGGCAATCTTTCGGAGACTTTCCACGGATTTTACA
AAAGCACCTACAGAACCAAGGAAGGGGAACTGAGGATACTAGCATCAAC
ACAATTTGAACCCACTGCAGCTAGAATGGCCTTTCCCTGCTTTGATGAA
CCTGCCTCCAAAGCAAGTTTCTCAATCAAAATTAGAAGAGAGCCAAGGC
ACCTAGCCATCTCCAATATGCCATTGGTGAAATCTGTGACTGTTGCTGA
AGGACTCATAGAAGACCATTTTGATGTCACTGTGAAGATGAGCACCTAT
CTGGTGGCCTTCATCATTTCAGATTTTGAGTCTGTCAGCAAGATAACCA
AGAGTGGAGTCAAGGTTTCTGTTTATGCTGTGCCAGACAAGATAAATCA
AGCAGATTATGCACTGGATGCTGCGGTGACTCTTCTAGAATTTTATGAG
GATTATTTCAGCATACCGTATCCCCTACCCAAACAAGATCTTGCTGCTA
TTCCCGACTTTCAGTCTGGTGCTATGGAAAACTGGGGACTGACAACATA
TAGAGAATCTGCTCTGTTGTTTGATGCAGAAAAGTCTTCTGCATCAAGT
AAGCTTGGCATCACAATGACTGTGGCCCATGAACTGGCTCACCAGTGGT
TTGGGAACCTGGTCACTATGGAATGGTGGAATGATCTTTGGCTAAATGA
AGGATTTGCCAAATTTATGGAGTTTGTGTCTGTCAGTGTGACCCATCCT
GAACTGAAAGTTGGAGATTATTTCTTTGGCAAATGTTTTGACGCAATGG
AGGTAGATGCTTTAAATTCCTCACACCCTGTGTCTACACCTGTGGAAAA
TCCTGCTCAGATCCGGGAGATGTTTGATGATGTTTCTTATGATAAGGGA
GCTTGTATTCTGAATATGCTAAGGGAGTATCTTAGTGCTGACGCATTTA
AAAGTGGTATTGTACAGTATCTCCAGAAGCATAGCTATAAAAATACAAA
AAACGAGGACCTGTGGGATAGTATGGCAAGTATTTGCCCTACAGATGGT
GTAAAAGGGATGGATGGCTTTTGCTCTAGAAGTCAACATTCATCTTCAT
CCTCACATTGGCATCAGGAAGGGGTGGATGTGAAAACCATGATGAACAC
TTGGACACTGCAGAAGGGTTTTCCCCTAATAACCATCACAGTGAGGGGG
AGGAATGTACACATGAAGCAAGAGCACTACATGAAGGGCTCTGACGGCG
CCCCGGACACTGGGTACCTGTGGCATGTTCCATTGACATTCATCACCAG
CAAATCCGACATGGTCCATCGATTTTCGCTAAAAACAAAAACAGATGTG
CTCATCCTCCCAGAAGAGGTGGAATGGATCAAATTTAATGTGGGCATGA
ATGGCTATTACATTGTGCATTACGAGGATGATGGATGGGACTCTTTGAC
TGGCCTTTTAAAAGGAACACACACAGCAGTCAGCAGTAATGATCGGGCG
AGTCTCATTAACAATGCATTTCAGCTCGTCAGCATTGGGAAGCTGTCCA
TTGAAAAGGCCTTGGATTTATCCCTGTACTTGAAACATGAAACTGAAAT
TATGCCCGTGTTTCAAGGTTTGAATGAGCTGATTCCTATGTATAAGTTA
ATGGAGAAAAGAGATATGAATGAAGTGGAAACTCAATTCAAGGCCTTCC
TCATCAGGCTGCTAAGGGACCTCATTGATAAGCAGACATGGACAGACGA
GGGCTCAGTCTCAGAGCGAATGCTGCGGAGTCAACTACTACTCCTCGCC
TGTGCGCACAACTATCAGCCGTGCGTACAGAGGGCAGAAGGCTATTTCA
GAAAGTGGAAGGAATCCAATGGAAACTTGAGCCTGCCTGTCGACGTGAC
CTTGGCAGTGTTTGCTGTGGGGGCCCAGAGCACAGAAGGCTGGGATTTT
CTTTATAGTAAATATCAGTTTTCTTTGTCCAGTACTGAGAAAAGCCAAA
TTGAATTTGCCCTCTGCAGAACCCAAAATAAGGAAAAGCTTCAATGGCT
ACTAGATGAAAGCTTTAANGGAGATAAAATAAAAACTCANGAGTTTCCA
CAAATTCTTACACTCATTGGNAGGAACCCAGTAGGCTACCCACTGGCCT
GGCAATTTCTGAGGAAAAACTGGAACAAACTTGTACAAAAGTTTGAACT
TGGCTCATCTTCCATAGCCCACATGGTAATGGGTACAACAAATCAATTC
TCCACAAGAACACGGCTTGAAGAGGTAAAAGGATTCTTCAGCTCTTTGA
AAGAAAATGGTTCTCAGCTCCGTTGTGTCCAACAGACAATTGAAACCAT
TGAAGAAAACATCGGTTGGATGGATAAGAATTTTGATAAAATCAGAGTG
TGGCTGCAAAGTGAAAAGCTTGAACGTATG
ERAP1 WT Protein sequence:
SEQ ID NO: 2
MVFLPLKWSLATMSFLLSSLLALLTVSTPSWCQSTEASPKRSDGTPFPW
NKIRLPEYVIPVHYDLLIHANLTTLTFWGTTKIEITASQPTSTIILHSH
HLQLSRATLRKGAGERPSEEPLQVLEHPRQEQIALLAPEPLLVGLPYTV
VIHYAGNLSETFHGFYKSTYRTKEGELRILASTQFEPTAARMAFPCFDE
PASKASFSIKIRREPRHLAISNMPLVKSVTVAEGLIEDHFDVTVKMSTY
LVAFIISDFESVSKITKSGVKVSVYAVPDKINQADYALDAAVTLLEFYE
DYFSIPYPLPKQDLAAIPDFQSGAMENWGLTTYRESALLDAEKSSASSK
LGITMTVAHELAHQWFGNLVTMEWWNDLWLNEGFAKFMEFVSVSVTHPE
LKVGDYFFGKCFDAMEVDALNSSHPVSTPVENPAQIREMFDDVSYDKGA
CILNMLREYLSADAFKSGIVQYLQKHSYKNTKNEDLWDSMASICPTDGV
KGMDGFCSRSQHSSSSSHWHQEGVDVKTMMNTWTLQKGFPLITTTVRGR
NVHMKQEHYMKGSDGAPDTGYLWHVPLTFITSKSDMVHRFSLKTKTDVL
ILPEEVEWIKFNVGMNGYYIVHYEDDGWDSLTGLLKGTHTAVSSNDRAS
LINNAFQLVSIGKLSIEKALDLSLYLKHETEIMPVFQGLNELIPMYKLM
EKRDMNEVETQFKAFLIRLLRDLIDKQTWTDEGSVSERMLRSQLLLLAC
AHNYQPCVQRAEGYFRKWKESNGNLSLPVDVTLAVFAVGAQSTEGWDFL
YSKYQFSLSSTEKSQIEFALCRTQNKEKLQWLLDESFXGDKIKTXEFPQ
ILTLDCRNPVGYPLAWQFLRKNWNKLVQKFELGSSSIAHMVMGTTNQFS
TRTRLEEVKGFFSSLKENGSQLRCVQQTIETIEENIGWMDKNFDKIRVW
LQSEKLERM

TABLE XIV
Identity and frequency of ERAP1 haplotypes in AS patients and
controls.

| | Frequency | | | | | | | | | | | | | | | | |
|---|---|---|---|---|---|---|---|---|---|---|---|---|---|---|---|---|---|
| | | Cases | | | | | | | | | | | | | | | |
| | Controls | (n = 34) | Amino acid at indicated position | | | | | | | | | | | | | | |
| | (n = 38) n (%) | n (%) | 82 | 102 | 115 | 127 | 199 | 349 | 528 | 575 | 581 | 725 | 727 | 730 | 737 | 752 | 874 |
| *001 | 8 (21) | 15 (44) | V | I | L | P | F | V | R | N | L | Q | L | E | V | R | M |
| *002 | 17 (44.5) | 1 (3) | I | L | P | R | S | M | K | D | S | R | L | Q | A | R | V |
| *003 | 1 (2.5) | 0 | I | L | P | R | S | M | K | D | S | R | L | Q | A | G | V |
| *004 | 0 | 1 (6) | I | L | P | R | S | M | K | D | S | R | A | Q | A | R | V |
| *005 | 4 (11) | 10 (29) | I | L | P | R | S | M | R | D | S | R | L | Q | A | R | V |
| *006 | 0 | 2 (6) | I | L | P | R | S | M | K | D | S | Q | L | E | A | R | V |
| *007 | 0 | 2 (6) | I | L | P | R | S | M | R | D | S | Q | L | Q | A | R | V |
| *008 | 1 (2.5) | 0 | I | L | P | R | S | M | R | D | S | R | L | E | A | R | V |
| *009 | 0 | 1 (3) | V | I | L | P | F | V | R | D | S | R | L | Q | A | R | V |
| *010 | 2 (5) | 0 | I | L | P | R | S | V | K | N | S | Q | L | Q | A | R | V |
| *011 | 4 (11) | 0 | V | I | L | R | F | M | R | D | L | R | L | E | V | R | V |
| *012 | 0 | 1 (3) | I | L | P | R | S | V | K | D | S | R | L | Q | A | R | V |
| *013 | 1 (2.5) | 0 | I | L | P | P | S | M | K | D | S | R | A | Q | A | R | V |

The most frequent haplotypes 001, 002, 005 and 011 are shown in bold. The difference between the cases and controls remains primarily at the haplotype combination level.

TABLE XV
Identity of ERAP1 allotype subtypes

| ERAP1 | Nucleotide at indicated position | | | | | | | | | | | | | | | | | |
|---|---|---|---|---|---|---|---|---|---|---|---|---|---|---|---|---|---|---|
| allotypes | 18 | 120 | 123 | 270 | 882 | 885 | 900 | 1338 | 1358 | 1359 | 1401 | 1405 | 1443 | 1470 | 1476 | 1497 | 2376 | 2490 |
| *001:01 | c | a | t | t | g | t | g | t | g | t | g | a | t | t | a | t | t | g |
| *001:02 | c | a | t | c | g | t | g | t | g | t | g | a | t | t | a | t | t | g |
| *001:03 | c | a | t | c | g | c | g | t | g | t | g | a | t | t | a | t | t | g |
| *001:04 | a | a | t | c | g | t | g | t | g | t | g | a | t | t | a | t | t | g |
| *001:05 | c | a | t | c | g | t | g | t | g | t | g | a | t | t | a | t | c | g |
| *001:06 | c | a | t | c | a | t | g | c | t | t | a | t | c | g | a | a | t | g |
| *001:07 | c | a | t | t | a | t | g | t | g | t | g | a | t | t | g | t | t | g |
| *002:01 | c | a | t | t | g | t | g | t | g | t | g | a | t | t | a | t | t | g |
| *002:02 | c | g | c | t | g | t | g | t | g | t | g | a | t | t | a | t | t | g |
| *005:01 | c | a | t | t | g | t | g | t | g | t | g | a | t | t | a | t | t | g |
| *005:02 | c | a | t | t | g | t | a | t | g | c | g | a | t | t | a | t | t | g |
| *005:03 | c | a | t | t | g | t | g | t | g | t | g | a | t | t | a | t | t | a |

TABLE XVI
Identity and frequency of ERAP1 haplotype combinations in
AS and control populations

Frequency

| Haplotype | Controls (n = 19) | Case (n = 17) |
|---|---|---|
| combination | n (%) | n (%) |
| 001 + 002 | 7 (37) | 0 |
| 002 + 005 | 3 (16) | 0 |
| 002 + 002 | 2 (11) | 0 |
| 002 + 011 | 2 (11) | 0 |
| 010 + 011 | 2 (11) | 0 |
| 001 + 008 | 1 (5) | 0 |
| 002 + 003 | 1 (5) | 0 |
| 005 + 013 | 1 (5) | 0 |
| 001 + 005 | 0 | 9 (53) |
| 001 + 001 | 0 | 2 (12) |
| 001 + 007 | 0 | 2 (12) |
| 002 + 006 | 0 | 1 (6) |
| 004 + 006 | 0 | 1 (6) |
| 005 + 009 | 0 | 1 (6) |
| 003 + 012 | 0 | 1 (6) |

TABLE XVII
New versus old haplotypes

| New nomenclature | Old nomenclature |
|---|---|
| ERAP1 haplotypes | ERAP1 haplotypes |
| *001 | 5SNP |
| *002 | WT |
| *003 | WT |
| *004 | WT |
| *005 | K528R |
| *006 | R725Q/Q730E |
| *007 | K528R/R725Q |
| *008 | K528R/Q730E |
| *009 | M349V/K528R |
| *010 | M349V/D575N/R725Q |
| *011 | K528R/Q730E |
| *012 | M349V |
| *013 | WT |

The additional haplotypes in the new nomenclature compared to the old come from the further stratification of the WT haplotype from one to four haplotypes;
WT = *002, *003, *004 and *013 haplotypes (this comes from examining SNPs from the entire sequence). In addition the K528R/Q730E haplotype is split into two;
K528R/Q730E = *008 and *011, again coming from examining other SNPs present in the entire sequence.

TABLE XVIII
Head and Neck Squamous Cell Carcinoma (HNSCC) data
Identity and frequency of ERAP1 haplotypes in HNSCC.

| | Frequency | | | | | | | | | | | | | | | | |
|---|---|---|---|---|---|---|---|---|---|---|---|---|---|---|---|---|---|
| | Cases | | | | | | | | | | | | | | | | |
| ERAP1 | (n = 36) n | Amino acid at indicated position | | | | | | | | | | | | | | | |
| haplotypes | (%) | 82 | 102 | 115 | 127 | 199 | 276 | 360 | 349 | 528 | 575 | 581 | 624 | 725 | 730 | 737 | 874 |
| *001 | 5 (14) | V | I | L | P | F | I | F | V | R | N | L | L | Q | E | V | M |
| *002 | 8 (22) | I | L | P | R | S | I | F | M | K | D | S | L | R | Q | A | V |
| *006 | 2 (6) | I | L | P | R | S | I | F | M | K | D | S | L | Q | E | A | V |
| *007 | 1 (3) | I | L | P | R | S | I | F | M | R | D | S | L | Q | Q | A | V |
| *011 | 3 (8) | V | I | L | R | F | I | F | M | R | D | L | L | R | E | V | V |
| *016 | 1 (3) | I | L | P | R | S | I | F | M | R | D | S | L | Q | E | V | V |
| *019 | 3 (8) | V | I | L | R | F | M | F | M | R | N | L | L | R | E | V | V |
| *020 | 2 (6) | V | I | L | R | F | I | F | M | K | D | L | L | R | E | V | V |
| *023 | 1 (3) | V | I | L | P | F | I | S | V | R | N | L | L | Q | E | V | M |
| *024 | 2 (6) | V | I | L | P | F | I | F | V | K | D | L | L | Q | E | A | V |
| *025 | 1 (3) | V | I | L | R | F | M | F | M | R | N | L | S | R | E | V | V |
| *027 | 2 (6) | V | I | L | P | F | I | F | V | R | N | L | L | R | E | V | M |
| *028 | 2 (6) | I | L | P | R | S | I | F | M | R | N | S | L | R | Q | A | V |
| *035 | 2 (6) | I | L | P | R | S | I | F | M | K | N | S | L | R | Q | A | V |
| *036 | 1 (3) | I | L | P | R | S | I | F | M | K | D | S | L | Q | Q | A | V |

Identity and frequency of ERAP1 haplotype combinations in HNSCC.

| Haplotype | Frequency |
|---|---|
| combination | Cases (n = 14) n (%) |
| 001 + 002 | 3 (21) |
| 002 + 035 | 2 (14) |
| 020 + 024 | 2 (14) |
| 027 + 028 | 2 (14) |
| 002 + 011 | 1 (7) |
| 002 + 019 | 1 (7) |
| 002 + 036 | 1 (7) |
| 007 + 019 | 1 (7) |
| 016 + 023 | 1 (7) |

TABLE XIX
Psoriasis
Identity and frequency of ERAP1 haplotypes in psoriasis

| | Frequency | | | | | | | | | | | | | |
|---|---|---|---|---|---|---|---|---|---|---|---|---|---|---|
| ERAP1 | Cases | Amino acid at indicated position | | | | | | | | | | | | |
| haplotypes | (n = 22) n (%) | 82 | 102 | 115 | 127 | 199 | 349 | 528 | 575 | 581 | 725 | 730 | 737 | 874 |
| *001 | 1 (5) | V | I | L | P | F | V | R | N | L | Q | E | V | M |
| *002 | 6 (27) | I | L | P | R | S | M | K | D | S | R | Q | A | V |
| *005 | 3 (14) | I | L | P | R | S | M | R | D | S | R | Q | A | V |
| *011 | 5 (23) | V | I | L | R | F | M | R | D | L | R | E | V | V |
| *012 | 7 (32) | I | L | P | R | S | V | K | D | S | R | Q | A | V |

Identity and frequency of ERAP1 allotype combinations in psoriasis.

| Haplotype | Frequency |
|---|---|
| combination | Cases (n = 8) n (%) |
| 002 + 005 | 2 (25) |
| 002 + 012 | 2 (25) |
| 001 + 002 | 1 (12.5) |
| 002 + 011 | 1 (12.5) |
| 005 + 011 | 1 (12.5) |
| 005 + 012 | 1 (12.5) |
| 011 + 012 | 1 (12.5) |

TABLE XX
Type-1-diabetes
Identity and frequency of ERAP1 haplotypes in Type-1-diabetes.

| | Frequency | | | | | | | | | | | | | | | | | |
|---|---|---|---|---|---|---|---|---|---|---|---|---|---|---|---|---|---|---|
| | Con- | | | | | | | | | | | | | | | | | |
| | trols | Cases | | | | | | | | | | | | | | | | |
| | (n = 16) | (n = 13) | Amino acid at indicated position | | | | | | | | | | | | | | | |
| | n (%) | n (%) | 82 | 102 | 115 | 127 | 199 | 276 | 346 | 349 | 528 | 575 | 581 | 725 | 727 | 730 | 737 | 874 |
| *001 | 3 (19) | 1 (8) | V | I | L | P | F | I | G | V | R | N | L | Q | L | E | V | M |
| *002 | 2 (12.5) | 4 (31) | I | L | P | R | S | I | G | M | K | D | S | R | L | Q | A | V |
| *005 | 0 | 2 (15) | I | L | P | R | S | I | G | M | R | D | S | R | L | Q | A | V |
| *008 | 0 | 1 (8) | I | L | P | R | S | I | G | M | R | D | S | R | L | E | A | V |
| *012 | 0 | 1 (8) | I | L | P | R | S | I | G | V | K | D | S | R | L | Q | A | V |
| *013 | 1 (6) | 0 | I | L | P | P | S | I | G | M | K | D | S | R | A | Q | A | V |
| *019 | 1 (6) | 0 | V | I | L | R | F | M | G | M | R | N | L | R | L | E | V | V |
| *022 | 1 (6) | 0 | V | I | L | P | F | M | G | V | R | D | L | R | L | E | V | M |
| *029 | 3 (19) | 2 (15) | V | I | L | R | F | I | D | V | R | D | L | R | L | E | V | M |
| *030 | 2 (12.5) | 0 | V | I | L | R | F | M | G | M | R | D | L | R | L | E | V | M |
| *031 | 2 (12.5) | 0 | V | I | L | R | F | I | G | M | K | D | L | R | L | Q | V | M |
| *032 | 0 | 1 (8) | V | I | L | P | F | M | G | M | K | D | S | R | L | Q | A | V |
| *033 | 0 | 1 (8) | V | I | L | P | F | M | G | M | R | D | L | R | L | E | V | M |
| *034 | 1 (6) | 0 | I | L | P | R | S | I | G | V | R | N | L | Q | L | E | V | M |

TABLE XXI
Identity and frequency of ERAP1 allotype combinations in
Type-1-diabetes

Frequency

| Haplotype | Controls (n = 5) | Case (n = 6) |
|---|---|---|
| combination | n (%) | n (%) |
| 001 + 002 | 1 (20) | 1 (17) |
| 002 + 034 | 1 (20) | 0 |
| 019 + 022 | 1 (20) | 0 |
| 029 + 030 | 1 (20) | 0 |
| 030 + 031 | 1 (20) | 0 |
| 002 + 005 | 0 | 1 (17) |
| 002 + 029 | 0 | 1 (17) |
| 005 + 012 | 0 | 1 (17) |
| 008 + 029 | 0 | 1 (17) |
| 032 + 033 | 0 | 1 (17) |

TABLE XXII
Response to NSAIDS

| | |
|---|---|
| | Frequency |
| | Response |
| Haplotype | to NSAIDS |

| combination | No (10) | Yes (7) |
|---|---|---|
| 001 + 005 | 5 | 4 |
| 001 + 001 | 0 | 2 |
| 001 + 007 | 1 | 1 |
| 002 + 006 | 1 | 0 |
| 004 + 006 | 1 | 0 |
| 005 + 009 | 1 | 0 |
| 003 + 012 | 1 | 0 |

There is a correlation for poor response to NSAIDS and the presence of hyperactive ERAP1 haplotypes (006 and 0007) shown in bold, in that the majority of AS individuals that have a hyperactive ERAP1 haplotype do not respond to NSAIDS. Thus the overall ERAP1 function helps to define the response to treatments, particularly a poor response to NSAIDS.

TABLE XXIII
New versus old haplotypes for HNSCC

| New | |
|---|---|
| nomenclature | |
| ERAP1 | |
| haplotypes | Old nomenclature ERAP1 haplotypes |
| *001 | 5SNP |
| *002 | WT |
| *006 | R725Q/Q730E |
| *007 | K528R/R725Q |
| *011 | K528R/Q730E |
| *016 | new haplotype = K528R/R725Q/Q730E (in table XI) |
| *019 | new haplotype = I276M/K528R/D575/Q730E (in table |
| | XI as K528R/D575N/Q730E) |
| *020 | new haplotype = Q730E (in table XI) |
| *023 | 5SNP (in table XI) |
| *024 | new haplotype = R127P/M349V/R725Q/Q730E (in table |
| | XI as M349V/R725Q/Q730E) |
| *025 | new haplotype = K528R/D575N/Q730E (in table XI) |
| *027 | new haplotype = R127P/M349V/K528R/D575N/Q730E |
| | (in table XI as M349V/K528R/D575N/Q730E) |
| *028 | new haplotype = K528R/D575N (in table XI) |
| *035 | new haplotype = D575N (in table XI) |
| *036 | new haplotype = R725Q (in table XI) |

TABLE XXIV
New versus old haplotypes for Type-1 Diabetes

| New | |
|---|---|
| nomenclature | |
| ERAP1 | |
| haplotypes | Old nomenclature ERAP1 haplotypes |
| *001 | 5SNP |
| *002 | WT |
| *005 | K528R |
| *008 | K528R/Q730E |
| *012 | M349V |
| *013 | WT |
| *019 | new haplotype = I276M/K528R/D575/Q730E |
| *022 | new haplotype = M349V/K528R/Q730E |
| *029 | K528/Q730E |
| *030 | K528/Q730E |
| *031 | WT |
| *032 | WT |
| *033 | K528/Q730E |
| *034 | 5SNP |

1. A method of diagnosing Ankylosing Spondylitis (AS), a spondyloarthropathy, arthritis, psoriasis, type-1 diabetes or a carcinoma comprising typing the ERAP1 haplotype of an individual to determine whether the individual has a hyper or hypo haplotype, wherein said haplotype comprises at least 2 SNP's.

2. A method according to claim 1:
wherein at least one or more of the following haplotypes are typed
R725Q/Q730E, K528R/R725Q and the 5SNP haplotype of Table III, and/or wherein at least one of the following five SNP's is typed I82V, L102I, P115L, S199F or S581L, and all four of M349V, K528R, R725Q, Q730E are typed, and optionally D575N is also typed.

3. A method according to claim 1 where:
said haplotype comprises 3, 4, 5 or more SNP's, and/or
2, 3 or all of the SNP's within the haplotype are at least 20 nucleotides apart from each other, and/or
said haplotype comprises at least 1, 2, 3, 4 or more SNP's at the positions shown in Table I, and/or
said haplotype comprises at least 1, 2, 3, 4 or more of the specific SNP's shown in Table I.

4. A method according to claim 1 comprising determining whether any of haplotypes 2 to 9 as shown in Table III are present in or absent from the genome of the individual, and optionally also determining whether any of the SNP's shown in Table VI are present or absent from the genome of the individual.

5. A method according to claim 1 comprising:
determining whether 3 or more, or all of the SNP's in any single row of Table III (excluding wild type) are present in or absent from the genome of the individual, and/or
determining whether 1, 2, 3, 4 or all of haplotypes 2 to 9 as shown in Table III are present in or absent from the genome of the individual, and/or
typing 3 or more, or all of the nucleotide positions at which the SNP's in Table I occur, and/or
typing both of the chromosomes of the individual at any of the nucleotide positions at which the SNP's in Table I occur.

6. A method according to claim 1 comprising determining whether any of the haplotypes shown in Table XIV, Table XV, Table XVI, Table XVII, XVIII, Table XIX, Table XX, Table XXI or Table XXII are present in or absent from the genome of the individual, wherein optionally the method is being carried out for diagnosis of the condition mentioned in the relevant Table.

7. A method according to claim 1 comprising contacting a specific binding agent with a polynucleotide from the individual and determining presence or absence of the haplotype based on whether or not binding to the polynucleotide occurs.

8. A method according to claim 7 wherein the specific binding agent:
is a polynucleotide, and/or
is provided in the form of a kit, and/or
is in the form of a gene array.

9. A method according to claim 1 which is carried out by analysis of ERAP1 protein of the individual.

10. A method according to claim 9 where said analysis comprises:
determining the presence of haplotype sequence directly in the ERAP1 protein, preferably by use of one or more specific antibodies, or
determining the presence of the haplotype by measuring the activity of the ERAP1 protein, preferably by measuring the trimming activity.

11. A method according to claim 9 comprising contacting a specific binding agent with ERAP1 protein from the individual and determining presence or absence of the based on whether or not binding to the ERAP1 protein occurs.

12. A method according to claim 11 wherein the specific binding agent is an antibody and/or is provided in the form of a kit.

13. A method according to claim 1 wherein the individual does not have any symptoms of any of the conditions listed in claim 1.

14. A method according to claim 1 which is carried out to diagnose AS, wherein the individual has back pain.

15. A method according to claim 1 which is carried out to diagnose the subset of AS, and optionally therapy for AS is chosen for the individual based on the diagnosis.

16. A therapeutic agent for AS for use in a method of treatment of a subset of AS in an individual, wherein said method comprises choosing said agent by the method of claim 15 and administering the chosen agent to the individual, and wherein said agent is preferably an analgesic, a non-steroidal anti-inflammatory drug, a corticosteroid or a disease modifying anti-rheumatic drug (DMARD).


SEQUENCE LISTING (30 sequences)

SEQ ID NO: 1 - DNA, 2823 bp, Homo sapiens
  CDS (1)..(2823)
  misc_feature (2469)..(2469): n is a, c, g, or t
atggtgtttctgcccctcaaatggtcccttgcaaccatgtcatttctactttcctcactgttggctctcttaactgtgtccactccttcatggtgtcagagcactgaagcatctccaaaacgtagtgatgggacaccatttccttggaataaaatacgacttcctgagtacgtcatcccagttcattatgatctcttgatccatgcaaaccttaccacgctgaccttctggggaaccacgaaaatagaaatcacagccagtcagcccaccagcaccatcatcctgcatagtcaccacctgcagctatctagggccaccctcaggaagggagctggagagaggccatcggaagaacccctgcaggtcctggaacacccccgtcaggagcaaattgcactgctggctcccgagcccctccttgtcgggctcccgtacacagttgtcattcactatgctggcaatctttcggagactttccacggattttacaaaagcacctacagaaccaaggaaggggaactgaggatactagcatcaacacaatttgaacccactgcagctagaatggcctttccctgctttgatgaacctgcctccaaagcaagtttctcaatcaaaattagaagagagccaaggcacctagccatctccaatatgccattggtgaaatctgtgactgttgctgaaggactcatagaagaccattttgatgtcactgtgaagatgagcacctatctggtggccttcatcatttcagattttgagtctgtcagcaagataaccaagagtggagtcaaggtttctgtttatgctgtgccagacaagataaatcaagcagattatgcactggatgctgcggtgactcttctagaattttatgaggattatttcagcataccgtatcccctacccaaacaagatcttgctgctattcccgactttcagtctggtgctatggaaaactggggactgacaacatatagagaatctgctctgttgtttgatgcagaaaagtcttctgcatcaagtaagcttggcatcacaatgactgtggcccatgaactggctcaccagtggtttgggaacctggtcactatggaatggtggaatgatctttggctaaatgaaggatttgccaaatttatggagtttgtgtctgtcagtgtgacccatcctgaactgaaagttggagattatttctttggcaaatgttttgacgcaatggaggtagatgctttaaattcctcacaccctgtgtctacacctgtggaaaatcctgctcagatccgggagatgtttgatgatgtttcttatgataagggagcttgtattctgaatatgctaagggagtatcttagtgctgacgcatttaaaagtggtattgtacagtatctccagaagcatagctataaaaatacaaaaaacgaggacctgtgggatagtatggcaagtatttgccctacagatggtgtaaaagggatggatggcttttgctctagaagtcaacattcatcttcatcctcacattggcatcaggaaggggtggatgtgaaaaccatgatgaacacttggacactgcagaagggttttcccctaataaccatcacagtgagggggaggaatgtacacatgaagcaagagcactacatgaagggctctgacggcgccccggacactgggtacctgtggcatgttccattgacattcatcaccagcaaatccgacatggtccatcgattttcgctaaaaacaaaaacagatgtgctcatcctcccagaagaggtggaatggatcaaatttaatgtgggcatgaatggctattacattgtgcattacgaggatgatggatgggactctttgactggccttttaaaaggaacacacacagcagtcagcagtaatgatcgggcgagtctcattaacaatgcatttcagctcgtcagcattgggaagctgtccattgaaaaggccttggatttatccctgtacttgaaacatgaaactgaaattatgcccgtgtttcaaggtttgaatgagctgattcctatgtataagttaatggagaaaagagatatgaatgaagtggaaactcaattcaaggccttcctcatcaggctgctaagggacctcattgataagcagacatggacagacgagggctcagtctcagagcgaatgctgcggagtcaactactactcctcgcctgtgcgcacaactatcagccgtgcgtacagagggcagaaggctatttcagaaagtggaaggaatccaatggaaacttgagcctgcctgtcgacgtgaccttggcagtgtttgctgtgggggcccagagcacagaaggctgggattttctttatagtaaatatcagttttctttgtccagtactgagaaaagccaaattgaatttgccctctgcagaacccaaaataaggaaaagcttcaatggctactagatgaaagctttaanggagataaaataaaaactcangagtttccacaaattcttacactcattggnaggaacccagtaggctacccactggcctggcaatttctgaggaaaaactggaacaaacttgtacaaaagtttgaacttggctcatcttccatagcccacatggtaatgggtacaacaaatcaattctccacaagaacacggcttgaagaggtaaaaggattcttcagctctttgaaagaaaatggttctcagctccgttgtgtccaacagacaattgaaaccattgaagaaaacatcggttggatggataagaattttgataaaatcagagtgtggctgcaaagtgaaaagcttgaacgtatg

SEQ ID NO: 2 - protein, 941 aa, Homo sapiens
  misc_feature (823)..(823): The ′Xaa′ at location 823 stands for Lys, or Asn.
MVFLPLKWSLATMSFLLSSLLALLTVSTPSWCQSTEASPKRSDGTPFPWNKIRLPEYVIPVHYDLLIHANLTTLTFWGTTKIEITASQPTSTIILHSHHLQLSRATLRKGAGERPSEEPLQVLEHPRQEQIALLAPEPLLVGLPYTVVIHYAGNLSETFHGFYKSTYRTKEGELRILASTQFEPTAARMAFPCFDEPASKASFSIKIRREPRHLAISNMPLVKSVTVAEGLIEDHFDVTVKMSTYLVAFIISDFESVSKITKSGVKVSVYAVPDKINQADYALDAAVTLLEFYEDYFSIPYPLPKQDLAAIPDFQSGAMENWGLTTYRESALLFDAEKSSASSKLGITMTVAHELAHQWFGNLVTMEWWNDLWLNEGFAKFMEFVSVSVTHPELKVGDYFFGKCFDAMEVDALNSSHPVSTPVENPAQIREMFDDVSYDKGACILNMLREYLSADAFKSGIVQYLQKHSYKNTKNEDLWDSMASICPTDGVKGMDGFCSRSQHSSSSSHWHQEGVDVKTMMNTWTLQKGFPLITITVRGRNVHMKQEHYMKGSDGAPDTGYLWHVPLTFITSKSDMVHRFSLKTKTDVLILPEEVEWIKFNVGMNGYYIVHYEDDGWDSLTGLLKGTHTAVSSNDRASLINNAFQLVSIGKLSIEKALDLSLYLKHETEIMPVFQGLNELIPMYKLMEKRDMNEVETQFKAFLIRLLRDLIDKQTWTDEGSVSERMLRSQLLLLACAHNYQPCVQRAEGYFRKWKESNGNLSLPVDVTLAVFAVGAQSTEGWDFLYSKYQFSLSSTEKSQIEFALCRTQNKEKLQWLLDESFXGDKIKTXEFPQILTLIGRNPVGYPLAWQFLRKNWNKLVQKFELGSSSIAHMVMGTTNQFSTRTRLEEVKGFFSSLKENGSQLRCVQQTIETIEENIGWMDKNFDKIRVWLQSEKLERM

SEQ ID NO: 3 - protein, 941 aa, Homo sapiens
  misc_feature (823)..(823): Xaa can be any naturally occurring amino acid
MVFLPLKWSLATMSFLLSSLLALLTVSTPSWCQSTEASPKRSDGTPFPWNKIRLPEYVIPVHYDLLIHANLTTLTFWGTTKIEITASQPTSTIILHSHHLQLSRATLRKGAGERPSEEPLQVLEHPRQEQIALLAPEPLLVGLPYTVVIHYAGNLSETFHGFYKSTYRTKEGELRILASTQFEPTAARMAFPCFDEPASKASFSIKIRREPRHLAISNMPLVKSVTVAEGLIEDHFDVTVKMSTYLVAFIISDFESVSKITKSGVKVSVYAVPDKINQADYALDAAVTLLEFYEDYFSIPYPLPKQDLAAIPDFQSGAMENWGLTTYRESALLFDAEKSSASSKLGITMTVAHELAHQWFGNLVTMEWWNDLWLNEGFAKFMEFVSVSVTHPELKVGDYFFGKCFDAMEVDALNSSHPVSTPVENPAQIREMFDDVSYDKGACILNMLREYLSADAFKSGIVQYLQKHSYKNTKNEDLWDSMASICPTDGVKGMDGFCSRSQHSSSSSHWHQEGVDVKTMMNTWTLQKGFPLITITVRGRNVHMKQEHYMKGSDGAPDTGYLWHVPLTFITSKSDMVHRFSLKTKTDVLILPEEVEWIKFNVGMNGYYIVHYEDDGWDSLTGLLKGTHTAVSSNDRASLINNAFQLVSIGKLSIEKALDLSLYLKHETEIMPVFQGLNELIPMYKLMEKRDMNEVETQFKAFLIRLLRDLIDKQTWTDEGSVSERMLRSQLLLLACAHNYQPCVQRAEGYFRKWKESNGNLSLPVDVTLAVFAVGAQSTEGWDFLYSKYQFSLSSTEKSQIEFALCRTQNKEKLQWLLDESFXGDKIKTXEFPQILTLIXRNPVGYPLAWQFLRKNWNKLVQKFELGSSSIAHMVMGTTNQFSTRTRLEEVKGFFSSLKENGSQLRCVQQTIETIEENIGWMDKNFDKIRVWLQSEKLERM

SEQ ID NO: 4 - DNA, 32 bp, Homo sapiens
gacgaattcatggtgtttctgcccctcaaatg

SEQ ID NO: 5 - DNA, 29 bp, Homo sapiens
gacctcgagcatacgttcaagcttttcac

SEQ ID NO: 6 - DNA, 26 bp, Homo sapiens
ctggtgctatggcaaactggggactg

SEQ ID NO: 7 - DNA, 26 bp, Homo sapiens
cagtccccagtttgccatagcaccag

SEQ ID NO: 8 - DNA, 34 bp, Artificial Sequence
  Modified human sequence
gcagtctgcagcgcgnnsagcatcatcaacttcg

SEQ ID NO: 9 - DNA, 34 bp, Artificial Sequence
  Modified human sequence
cgaagttgatgatgctsnncgcgctgcagactgc

SEQ ID NO: 10 - protein, 5 aa, Homo sapiens
AIVMK

SEQ ID NO: 11 - protein, 6 aa, Homo sapiens
LEQLEK

SEQ ID NO: 12 - DNA, 35 bp, Homo sapiens
gaccggtttgtatgctaaaagtatcattaatttcg

SEQ ID NO: 13 - DNA, 35 bp, Homo sapiens
cgaaattaatgatacttttagcatacaaaccggtc

SEQ ID NO: 14 - DNA, 38 bp, Homo sapiens
ctatcattaatttcgaacatcttaaatgcggtgctagc

SEQ ID NO: 15 - DNA, 38 bp, Homo sapiens
gctagcaccgcatttaagatgttcgaaattaatgatag

SEQ ID NO: 16 - DNA, 39 bp, Homo sapiens
cattaatttcgaacatcttaaatgcggtgctagcggtgg

SEQ ID NO: 17 - DNA, 39 bp, Homo sapiens
ccaccgctagcaccgcatttaagatgttcgaaattaatg

SEQ ID NO: 18 - protein, 8 aa, Homo sapiens
SIINFEHL

SEQ ID NO: 19 - protein, 5 aa, Homo sapiens
GAMEN

SEQ ID NO: 20 - DNA, 22 bp, Homo sapiens
cttggcatcacagtgactgtgg

SEQ ID NO: 21 - DNA, 22 bp, Homo sapiens
ccacagtcactgtgatgccaag

SEQ ID NO: 22 - DNA, 26 bp, Homo sapiens
ggacactgcagaggggctttcctctg

SEQ ID NO: 23 - DNA, 26 bp, Homo sapiens
cagaggaaagcccctctgcagtgtcc

SEQ ID NO: 24 - DNA, 23 bp, Homo sapiens
cagcaaatccaacatggtccatc

SEQ ID NO: 25 - DNA, 23 bp, Homo sapiens
gatggaccatgttggatttgctg

SEQ ID NO: 26 - DNA, 28 bp, Homo sapiens
gctcagtctcagagcaaatgctgcggag

SEQ ID NO: 27 - DNA, 28 bp, Homo sapiens
ctccgcagcatttgctctgagactgagc

SEQ ID NO: 28 - DNA, 23 bp, Homo sapiens
gctgcggagtgaactactactcc

SEQ ID NO: 29 - DNA, 23 bp, Homo sapiens
ggagtagtagttcactccgcagc

SEQ ID NO: 30 - protein, 8 aa, Homo sapiens
IINFEHLK